(19) United States
(12) Patent Application Publication
(10) Pub. No.: US 2005/0166271 A1
(43) Pub. Date: Jul. 28, 2005

(54) Fatty acid desaturase gene obtained from pomegranate and method for the production of unsaturated fatty acids

(76) Inventors: Ivo Feubner, Halle (DE); Ellen Hornung, Quedlinburg (DE); Christian Pernstich, Halle (DE); Andreas Renz, Limburgerhof (DE)

Correspondence Address: CONNOLLY BOVE LODGE & HUTZ, LLP, P O BOX 2207, WILMINGTON, DE, 19899, US

(21) Appl. No.: 10/484,202

(22) PCT Filed: Jul. 9, 2002
(86) PCT No.: PCT/EP02/07611

(30) Foreign Application Priority Data
Jul. 20, 2001 (DE) 101 34 660.3

(51) Int. Cl. C12Q 1/68; A01K 67/00; C07H 21/04; C12N 9/04; A01H 1/00; C12N 5/04

(52) U.S. Cl. 800/8; 800/281; 435/6; 435/69.1; 435/190; 435/320.1; 435/325; 435/419; 435/468; 536/23.2

(57) ABSTRACT

The present invention relates to a process for the production of unsaturated or saturated fatty acids and to a process for the production of oils and/or triglycerides with an increased content of unsaturated or saturated fatty acids. The invention furthermore relates to nucleic acid sequences, nucleic acid constructs, vectors and organisms comprising the nucleic acid sequences, nucleic acid constructs and/or vectors. Moreover, the invention relates to fatty acid mixtures and to triglycerides with an increased content of unsaturated fatty acids, and to their use.

The present invention relates to a process for the production of unsaturated or saturated fatty acids and to a process for the production of oils and/or triglycerides with an increased content of unsaturated or saturated fatty acids.

The invention furthermore relates to nucleic acid sequences, nucleic acid constructs, vectors and organisms comprising the nucleic acid sequences, nucleic acid constructs and/or vectors. Moreover, the invention relates to fatty acid mixtures and to triglycerides with an increased content of unsaturated fatty acids and to their use.

Fatty acids and triglycerides have a multiplicity of uses in the food industry, in animal nutrition, in cosmetics and in the pharmacological sector. Depending on whether they are free saturated or unsaturated fatty acids or triglycerides with an increased content of saturated or unsaturated fatty acids, they are suitable for a wide range of uses; thus, for example, polyunsaturated fatty acids are added to infant formula to increase the nutritional value. The various fatty acids and triglycerides are obtained mainly from microorganisms such as Mortierella or Schizochytrium, or from oil-producing plants such as soybeans, oilseed rape, sunflower and others, and are, as a rule, obtained in the form of their triacylglycerides. However, they are also advantageously obtained from animals such as fish. The free fatty acids are advantageously produced by hydrolysis.

Depending on the intended use, oils with saturated or unsaturated fatty acids are preferred; thus, for example, lipids with unsaturated fatty acids, specifically polyunsaturated fatty acids, are preferred for human nutrition since they have a positive effect on the cholesterol level in the blood and thus on the possibility of heart disease. They are used in a wide range of dietetic foods or medicaments.

Especially valuable and sought-after unsaturated fatty acids are what are known as conjugated unsaturated fatty acids, such as conjugated linoleic acid. A series of positive effects have been identified for conjugated fatty acids; thus, the administration of conjugated lineoleic acid reduces the body fat in humans and animals, or increases the rate at which feed is converted into body weight in the case of animals (WO 94/16690, WO 96/06605, WO 97/46230, WO 97/46118). The administration of conjugated linoleic acid also has a positive effect on, for example, allergies (WO 97/32008) or cancer (Banni et al., Carcinogenesis, Vol. 20, 1999: 1019-1024, Thompson et al., Cancer, Res., Vol. 57, 1997: 5067-5072).

The chemical production of conjugated fatty acids, for example punicic acid or conjugated linoleic acid, is described in U.S. Pat. No. 3,356,699 and U.S. Pat. No. 4,164,505. Punicic acid occurs naturally in Punica granatum (El-Shaarawy and Nahapetian, Fette Seifen Anstrichmittel, 85, 1983: 123-126; Melgarejo et al., Sci. Food Agric., 69, 1995, 253-256; Melgarejo and Artés, Journal of the Science of Food and Agriculture, 2000, 80, 1452-1454). Conjugated linoleic acid is found, for example, in beef (Chin et al., Journal of Food Composition and Analysis, 5, 1992: 185-197), in milk (Dhiman et al., Journal of Dairy Science, 1999, 82, 2146-56) and milk products.

Not biochemical studies into the biosynthesis of punicic acid are available; however, the biosynthesis of calendulic acid has been the subject of such studies (Crombie et al., J. Chem. Soc. Chem. Commun., 15, 1984: 953-955 and J. Chem. Soc. Perkin Trans., 1, 1985: 2425-2434; Fritsche et al., FEBS Letters, 462, 1999, 249-253; Cahoon et al., J. Biol. Chem., 276, 2001, 2637-2643; Qiu et al., Plant Physiology, 125, 2001, 847-855). According to these studies, conjugated fatty acids such as calendulic acid, eleostearic acid or punicic acid are biosynthesized via the desaturation of oleic acid to linoleic acid by means of a Δ-12-desaturase and a further desaturation step, in conjunction with a rearrangement of double bond to the conjutrienoic fatty acid by a specific conjutriene-forming desaturase. Qiu et al., (Plant Physiology, 125, 2001, 847-855) describe not only the preparation of conjugated calendulic acid, but also the preparation of conjugated linoleic acid by the enzymatic activity of desaturase. However, the disadvantage of this secondary activity is that the enzymatic action gives rise to the undesired 8,10-isomer of the conjugated linoleic acid.

Due to their positive characteristics, there have been no lack of attempts in the past to make available genes involved in fatty acid or triglyceride synthesis for the production, in a variety of organisms, of oils with a modified content of unsaturated fatty acids. Thus, WO 91/13972 and its US equivalent describe a Δ-9-desaturase. WO 93/11245 describes a Δ-15-desaturase, while WO 94/11516 describes a Δ-12-desaturase. Δ-6-Desaturases are described in WO 93/06712 and WO 96/21022. Other desaturases are described, for example, in EP-A-0 550 162, WO 94/18337, WO 97/30582, WO 97/21340, WO 95/18222, EP-A-0 794 250, Stukey et al., J. Biol. Chem., 265, 1990: 20144-20149, Wada et al., Nature 347, 1990: 200-203 or Huang et al., Lipids 34, 1999: 649-659. However, the various desaturases have only been subjected to insufficient biochemical characterization since the enzymes, being membrane-bound proteins, can be isolated and characterized only with great difficulty (McKeon et al., Methods in Enzymol. 71, 1981: 12141-12147, Wang et al., Plant Physiol. Biochem., 26, 1988: 777-792).

In yeasts, both a shift of the fatty acid spectrum toward unsaturated fatty acids and an increase in the productivity has been detected (see Huang et al., Lipids 34, 1999: 649-659, Napier et al., Biochem. J., Vol. 330, 1998: 611-614). However, the expression of the various desaturases in transgenic plants was not as successful as desired. While a shift of the fatty acid spectrum toward unsaturated fatty acids was demonstrated, it emerged that the synthesis rate of transgenic plants decreased greatly, i.e. smaller amounts of oils were isolated than in the original plants.

There therefore remains a great demand for novel genes which encode enzymes which are involved in the biosynthesis of unsaturated fatty acids and which make possible the synthesis of the latter, and specifically of conjugated unsaturated fatty acids, and their production on an industrial scale.

It is an object of the present invention to provide further desaturases for the synthesis of unsaturated conjugated fatty acids. We have found that this object is achieved by an isolated nucleic acid sequence which encodes a polypeptide with desaturase activity, selected from the group consisting of:
a) a nucleic acid sequence with the sequence shown in SEQ ID NO: 2 or SEQ ID NO: 7, or
b) nucleic acid sequences which are derived from the amino acid sequence shown in SEQ ID NO: 3 or SEQ ID NO: 8 by back translation into a nucleic acid sequence, owing to the degeneracy of the genetic code, or
c) derivatives of the nucleic acid sequence shown in SEQ ID NO: 2 or SEQ ID NO: 7 which encode polypeptides with the amino acid sequences shown in SEQ ID NO: 3 or SEQ ID NO: 8 and which have at least 75% identity at the amino acid level, without the enzymatic action of the polypeptides being substantially modified.

A further embodiment of the invention is a process for the production of oils or triglycerides with an increased content of unsaturated fatty acids, which comprises the following process steps:
a) introducing at least one above-described nucleic acid sequence of the invention, at least one nucleic acid construct of the invention or at least one vector of the invention (both described hereinbelow) into an oil-producing organism;
b) culturing this organism; and
c) isolating the oil present in the organism or the triglycerides present in the organism.

After the fatty acids present in the oil or the triglycerides have been isolated, they can be liberated by the methods of acid or alkaline hydrolysis, with which the skilled worker is familiar.

It is advantageous for the above-described process according to the invention additionally to introduce, in process step (a), at least one further nucleic acid into the organism, which nucleic acid encodes a polypeptide with desaturase activity selected from the group consisting of:
a) a Δ-5-desaturase, a Δ-6-desaturase, a Δ-8-desaturase or Δ-12-desaturase, or
b) a nucleic acid sequence with the sequence shown in SEQ ID NO: 5, or
c) nucleic acid sequences which are derived from the amino acid sequence shown in SEQ ID NO: 6 by back translation into a nucleic acid sequence, owing to the degeneracy of the genetic code, or
d) derivatives of the nucleic acid sequence shown in SEQ ID NO: 5 which encode polypeptides with the amino acid sequences shown in SEQ ID NO: 6 and which have at least 90% identity at the amino acid level, without the enzymatic action of the polypeptides being substantially modified.

Besides the abovementioned punicic acid desaturase (=SEQ ID NO: 2 and SEQ ID NO: 7, for the purpose of the present invention, the singular is also meant to include the plural), a further Δ-12-desaturase, such as the Δ-12-desaturase of SEQ ID NO: 5, is advantageous for the above-described process for the production of oils and/or triglycerides with an increased content of unsaturated fatty acids, for example unsaturated conjugated fatty acids such as punicic acid, for example from oleic acid.

The formation of conjutrienoic fatty acids, for example from oleic acid, is especially advantageous since oilseed crops such as oilseed rape would make possible an inexpensive and simple availability of conjutriene owing to their high oleic acid content. Since, however, their linoleic acid content is low (Mikoklajczak et al., Journal of the American Oil Chemical Society, 38, 1961, 678-81), the use of the abovementioned Δ-12-desaturases is advantageous for the production of linoleic acid.

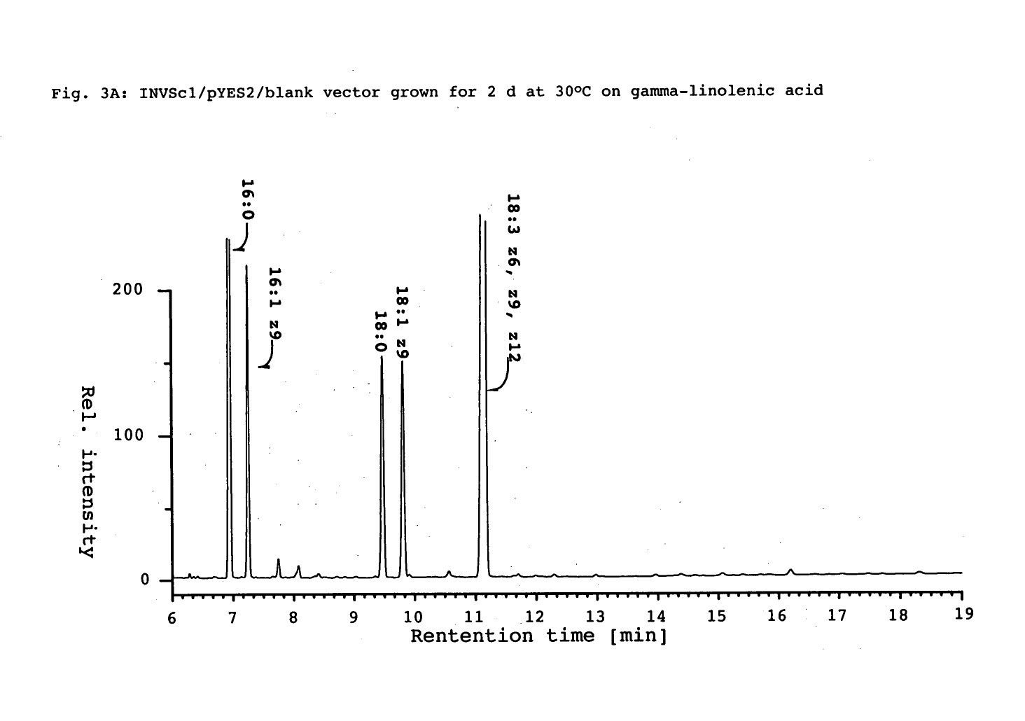

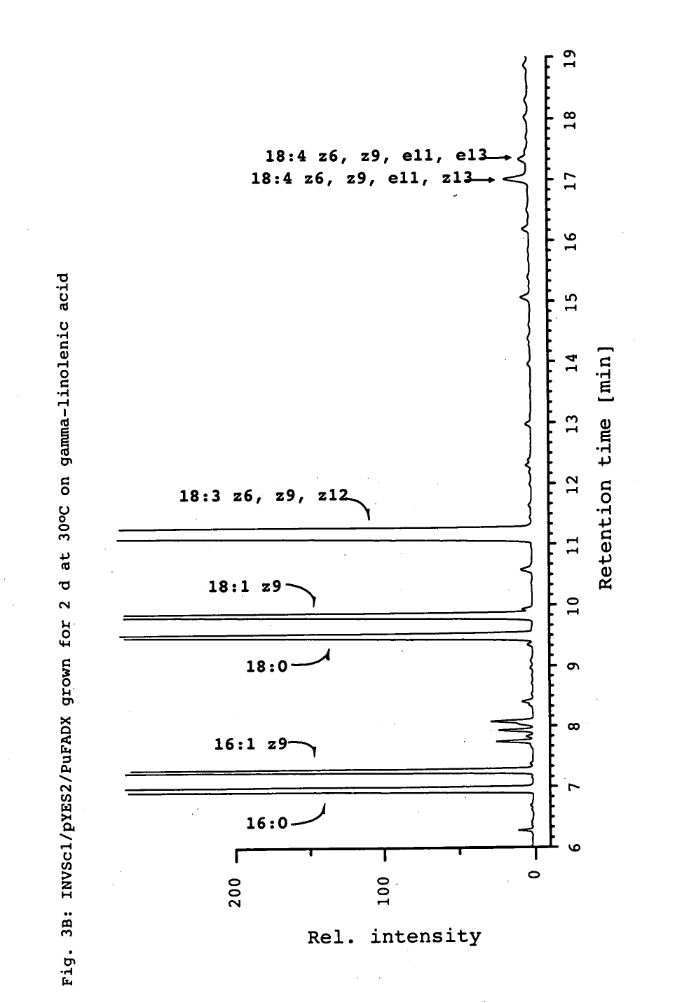

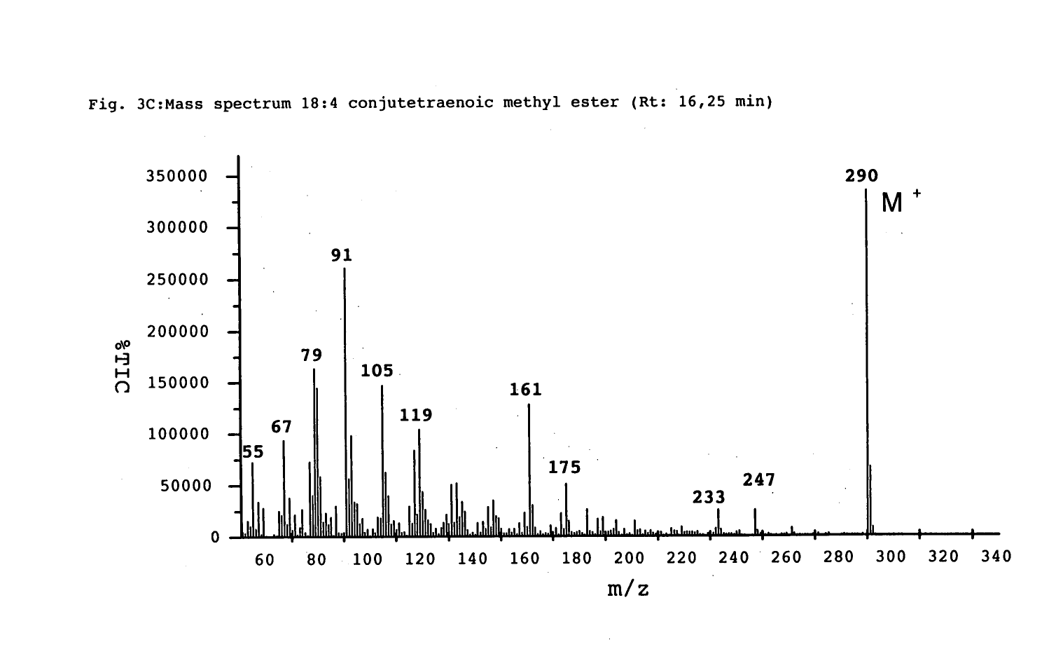

A derivative, or derivatives, is understood as meaning, for example, functional homologs of the enzyme encoded by SEQ ID NO: 2, SEQ ID NO: 5 or SEQ ID NO: 7 or the enzymatic activity of this enzyme, i.e. enzymes which catalyze the same enzymatic reactions as the enzyme encoded by SEQ ID NO: 3, SEQ ID NO: 5 or SEQ ID NO: 7. These genes also make possible the advantageous production of unsaturated conjugated fatty acids. Unsaturated fatty acids are understood hereinbelow as meaning mono- and polyunsaturated fatty acids whose double bonds may be conjugated or else unconjugated. The sequences shown in SEQ ID NO: 2 or SEQ ID NO: 7 encode novel unknown desaturases which are involved in the synthesis of punicic acid in Punica granatum. The enzymes preferentially convert (9Z,12Z) octadecadienoic/linoleic acid into (9Z,11E,13Z)octadeca-conjutrienoic/punicic acid. They are termed punicic acid desaturase(s) hereinbelow. Further substrates of these enzymes are, for example, γ-linolenic acid, which is converted into a variety of 18:4-conjutetrenoic acid isomers (FIG. 3B). Oleic acid too is converted by the enzymes. 9_{cis}11ₜᵣₐₙₛ-conjugated linoleic acid is advantageously formed.

The nucleic acid sequences according to the invention or fragments thereof can be used advantageously for isolating further genomic sequences via homology screening.

The abovementioned derivatives can be isolated for example from other organisms eukaryotic organisms like plants such as Calendula stellata, Osteospermum spinescens or Osteospermum hyoseroides, algae, dinoflagellates or fungi.

Derivatives or functional derivatives of the sequence mentioned in SEQ ID NO: 2, SEQ ID NO: 5 or SEQ ID NO: 7 are furthermore understood as meaning for example, allelic variants which, in the case of SEQ ID NO: 2 or SEQ ID NO: 7, have at least 75% homology, preferably at least 80% homology, especially preferably at least 85% homology, very especially preferably 90% homology, at the derived amino acid level. In the case of SEQ ID NO: 5, the derivatives have 90%, preferably 95%, especially preferably 98%, homology. The homology was calculated over the entire amino acid region. The program PileUP was used (J. Mol. Evolution., 25, 351-360, 1987, Higgins et al., CABIOS, 5 1989: 151-153). For the purposes of the present invention, homology is understood as meaning identity. The two terms are synonymous. The amino acid sequences derived from the abovementioned nucleic acid sequences can be seen from sequence SEQ ID NO: 3, SEQ ID NO: 6 or SEQ ID NO: 8. Allelic variants encompass in particular functional variants which are obtainable from the sequence shown in SEQ ID NO: 1 by deletion, insertion or substitution of nucleotides, the enzymatic activity of the derived synthesized proteins being retained.

Such DNA sequences can be isolated from other eukaryotes as mentioned above starting from the DNA sequence described in SEQ ID NO: 2, SEQ ID NO: 5 or SEQ ID NO: 7 or parts of these sequences, for example using customary hybridization methods or the PCR technique. These DNA sequences hybridize with the abovementioned sequences under standard conditions. Oligonucleotides which are advantageously used for the hybridization are short oligonucleotides, for example of the conserved regions, which can be identified in a manner known to the skilled worker by a comparison with other desaturase genes. However, longer fragments of the nucleic acids according to the invention, or the complete sequences, may also be used for the hybridization. Depending on the nucleic acid used—oligonucleotides, longer fragment or complete sequence—or depending on the type of nucleic acid—DNA or RNA—which are used for the hybridization, these standard conditions vary. Thus, for example, the melting temperatures of DNA:DNA hybrids are approx. 10° C. lower than those of equally long DNA:RNA hybrids.

Depending on the nucleic acid, standard conditions are understood as meaning, for example, temperatures of between 42 and 58° C. in an aqueous buffer solution with a concentration of between 0.1 and 5×SSC (1×SSC=0.15 M NaCl, 15 mM sodium citrate, pH 7.2) or additionally in the presence of 50% formamide such as, for example, 42° C. in 5×SSC, 50% formamide. The hybridization conditions for DNA:DNA hybrids are advantageously 0.1×SSC and temperatures between approximately 20° C. and 45° C., preferably between approximately 30° C. and 45° C. For DNA:RNA hybrids, the hybridization conditions are advantageously 0.1×SSC and temperatures between approximately 30° C. and 55° C., preferably between approximately 45° C. and 55° C. These temperatures stated for the hybridization are melting temperature values which have been calculated by way of example for a nucleic acid with a length of approx. 100 nucleotides and a G+C content of 50% in the absence of formamide. The experimental conditions for DNA hybridization are described in specialist textbooks of genetics such as, for example, Sambrook et al., “Molecular Cloning”, Cold Spring Harbor Laboratory, 1989, and can be calculated using formulae with which the skilled worker is familiar, for example as a function of the length of the nucleic acids, the nature of the hybrids or the G+C content. The skilled worker can find more information on hybridization in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Moreover, derivatives are understood as meaning homologs of the sequences SEQ ID NO: 2, SEQ ID NO: 5 or SEQ ID NO: 7, for example eukaryotic homologs, truncated sequences, single-stranded DNA of the coding and noncoding DNA sequence or RNA of the coding and noncoding DNA sequence.

Homologs of the sequences SEQ ID NO: 2, SEQ ID NO: 5 or SEQ ID NO: 7 are furthermore understood as meaning derivatives such as, for example, promoter variants. These variants can be modified by one or more nucleotide substitutions, by insertion(s) and/or deletion(s) without, however, the functionality or efficacy of the promoters being adversely affected. Moreover, the promoters can be increased in their efficacy by modifying their sequence, or they can be replaced completely by more effective promoters, including promoters of heterologous organisms.

Derivatives are also advantageously understood as meaning variants whose nucleotide sequence in the region −1 to −2000 upstream of the start codon has been modified in such a way that gene expression and/or protein expression is modified, preferably increased. Moreover, derivatives are also understood as meaning variants whose 3′ end has been modified.

It is advantageous for an optimal expression of heterologous genes in organisms to modify the nucleic acid sequences in accordance with the specific codon usage of the organism. The codon usage can be determined readily with the aid of computer evaluations of other, known genes of the organism in question.

The punicic acid desaturase gene can be combined advantageously in the process according to the invention with further genes of fatty acid biosynthesis. Particularly advantageous is the combination with the Δ-12-desaturase stated under SEQ ID NO: 5 and 6. Further advantageous sequences are desaturase sequences such as Δ-5-desaturase, Δ-6-desaturase or Δ-8-desaturase sequences, acetyltransferase sequences or elongase sequences.

The amino acid sequences according to the invention are understood as meaning proteins comprising an amino acid sequence shown in SEQ ID NO: 3, SEQ ID NO: 6 or SEQ ID NO: 8 or a sequence which can be obtained therefrom by substitution, inversion, insertion or deletion of one or more amino acid residues, the enzymatic activity of the proteins shown in SEQ ID NO: 3, SEQ ID NO: 6 or SEQ ID NO: 8 being retained, or not modified substantially. These proteins which are not substantially modified are therefore still enzymatically active, i.e. functional. The term not essentially modified is understood as meaning all those enzymes which retain at least 10%, preferably 20%, especially preferably 30% of the enzymatic activity of the original enzyme, or whose enzymatic activity is increased by at least 10%, preferably by 50%, especially preferably by at least 100% in comparison with the original enzyme, or the original amino acid sequence. In this context, specific amino acids may be replaced for example by those with similar physicochemical properties (spatial arrangement, basicity, hydrophobicity and the like). For example, arginin residues are exchanged for lysin residues, valin residues for isoleucin residues or aspartic acid residues for glutamic acid residues. However, it is also possible for the sequence of one or more amino acids to be exchanged or for one or more amino acids to be added or removed, or several of these measures may be combined with each other.

The nucleic acid constructs or fragments according to the invention are understood as meaning the sequences stated in SEQ ID NO: 2, SEQ ID NO: 5 or SEQ ID NO: 7, sequences which are the result of the genetic code and/or their functional or nonfunctional derivatives, which have advantageously been linked operably to one or more regulatory signals in order to increase gene expression. For example, these regulatory sequences are sequences to which inductors or repressors bind, thus regulating the expression of the nucleic acid. In addition to these new regulatory sequences, or instead of these sequences, the natural regulation of these sequences before the actual structural genes may still be present and may, if appropriate, have been modified genetically so that natural regulation has been disabled and the expression of the genes increased. However, the gene construct (=nucleic acid construct) may also be simpler in construction, i.e. no additional regulatory signals were inserted before the sequence or its derivatives, and the natural promoter together with its regulation was not removed. Instead, the natural regulatory sequence was mutated in such a way that regulation no longer takes place and gene expression is increased. These modified promoters may also be positioned alone before the natural gene in order to increase the activity. Moreover, the gene construct may advantageously also comprise one or more what are known as enchancer sequences linked operably to the promoter, and these enchancer sequences make possible an increased expression of the nucleic acid sequence. Also, additional advantageous sequences may be inserted at the 3′ end of the DNA sequences, such as further regulatory elements or terminators. One or more copies of the calendulic acid desaturase gene may be present in the gene construct.

Advantageous regulatory sequences for the process according to the invention are present, for example, in promoters such as the cos, tac, trp, tet, trp-tet, lpp, lac, lpp-lac, lacI^{q,} T7, T5, T3, gal, trc, ara, SP6, λ-P_{R} or the λ-P_{L} promoter, all of which are advantageously used in Gram-negative bacteria. Further advantageous regulatory sequences are present in, for example, the Gram-positive promoters amy and SPO2, in the yeast or fungal promoters ADC1, MFα, AC, P-60, CYC1, GAPDH, TEF, rp28, ADH or in the plant promoters such as CaMV/35S [Franck et al., Cell 21(1980) 285-294], PRP1 (Ward et al., Plant. Mol. Biol. 22 (1993)], SSU, OCS, lib4, STLS1, B33, nos or in the ubiquitin promoter. Further advantageous plant promoters are, for example, a benzenesulfonamide-inducible promoter (EP 388186), a tetracyclin-inducible promoter (Gatz et al., (1992) Plant J. 2, 397-404), an abscisic-acid-inducible promoter (EP335528) or an ethanol- or cyclohexanone-inducible promoter (WO 93/21334). Further plant promoters are, for example, the potato cytosolic FBPase promoter, the potato ST-LSI promoter (Stockhaus et al., EMBO J. 8 (1989) 2445-245), the Glycine max phosphoribosylpyrophosphate amidotransferase promoter (see also Genbank Accession Number U87999), or a node-specific promoter as may be described in EP 249676. Advantageous plant promoters are in particular those which ensure the expression in tissues or plant parts in which fat biosynthesis or its precursors take place. Promoters which may be mentioned in particular are those which ensure seed-specific expression, such as, for example, the usp promoter, the LEB4 promoter, the phaseolin promoter or the napin promoter.

In principle, all natural promoters with their regulatory sequences, like those mentioned above, may be used for the process according to the invention. In addition, synthetic promoters may also be advantageously used.

The nucleic acid fragment(s) [=gene construct(s), nucleic acid construct(s)] may additionally comprise further genes to be introduced into the organisms, as described above. These genes may be subject to separate regulation or to the same regulatory region as the desaturase genes according to the invention. These genes are, for example, further biosynthesis genes, advantageously biosynthesis genes of fatty acid biosynthesis, which make possible an increased biosynthesis rate. Examples which may be mentioned are the genes for Δ-15-, Δ-12-, Δ-9-, Δ-6-, and Δ-5-desaturase, the various hydroxylases, acetylenase, the acyl-ACP thioesterases, β-ketoacyl-ACP synthases or β-ketoacyl-ACP reductases. It is advantageous to use the desaturase genes in the same nucleic acid construct, preferably the Δ-12-desaturase gene, as shown in SEQ ID NO: 5 and 6.

For expression in a host organism, for example a microorganism such as a fungus or a plant, the nucleic acid constructs according to the invention are advantageously inserted into a vector such as, for example, a plasmid, a phage or other DNA, which makes possible optimal expression of the genes in the host. Examples of suitable plasmids in E. coli are pLG338, pACYC184, pBR322, pUC18, pUC19, pKC30, pRep4, pHS1, pHS2, pPLc236, pMBL24, pLG200, pUR290, pIN-III¹¹³-B1, λgt11 or pBdCI, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2∝M, pAG-1, YEp6, YEp13 or pEMBLYe23 or in plants pLGV23, pGHlac⁺, pBIN19, pAK2004, pVKH or pDH51, or derivatives of the abovementioned plasmids. The plasmids mentioned constitute a small selection of the plasmids which are possible. Further plasmids are well known to the skilled worker and can be found, for example, in the book Cloning Vectors (Eds. Pouwels P. H. et al. Elsevier, Amsterdam-New York-Oxford, 1985, ISBN 0 444 904018). Suitable plant vectors are described, inter alia, in “Methods in Plant Molecular Biology and Biotechnology” (CRC Press), Chapter 6/7, pp.71-119.

In addition to plasmids, vectors are also understood as meaning all of the other vectors known to the skilled worker such as, for example, phages, viruses such as SV40, CMV, baculovirus, adenovirus, transposons, IS elements, phasmids, phagemids, cosmids, linear DNA or circular DNA. These vectors can be replicated autonomously in the host organism or else chromosomally; chromosomal replication is preferred.

The vector advantageously comprises at least one copy of the nucleic acid sequences according to the invention and/or the nucleic acid fragments according to the invention.

To increase the repetition frequency of the genes, the nucleic acid sequences or homologous genes can be incorporated for example into a nucleic acid fragment or into a vector which preferably comprises the regulatory gene sequences assigned to the genes in question, or analogously acting promoter activity. Those regulatory sequences which increase gene expression are used in particular.

The nucleic acid fragments for the expression of the other genes which are present advantageously additionally comprise 3′- and/or 5′-terminal regulatory sequences for increasing expression, the sequences being selected for optimal expression as a function of the host organism chosen and the gene or genes.

These regulatory sequences are intended to make possible the directed expression of the genes and protein expression. Depending on the host organism, this may mean, for example, that the gene is expressed and/or overexpressed only after it has been induced, or that it is expressed and/or overexpressed immediately.

In this context, the regulatory sequences or factors can preferably have a positive effect on, and thus increase, the gene expression of the genes introduced. Thus, an enhancement of the regulatory elements can advantageously take place at the transcription level by using strong transcription signals such as promoters and/or enhancers. In addition, however, an enhanced translation is also possible, for example by improving the stability of the mRNA.

In a further embodiment of the vector, the gene construct according to the invention (in the following text, the singular is also understood as encompassing the plural) can advantageously also be introduced into the organisms in the form of a linear DNA and integrated into the genome of the host organism via heterologous or homologous recombination. This linear DNA may be composed of a linearized plasmid or else only of the nucleic acid fragment as vector or of the nucleic acid sequence according to the invention.

The nucleic acid sequence according to the invention (in the following text, the singular is also to be understood as encompassing the plural) is advantageously cloned into a nucleic acid construct together with at least one reporter gene, and this nucleic acid construct is introduced into the genome. This reporter gene should make possible easy detectability via a growth assay, fluorescence assay, chemoluminescence assay, bioluminescence assay or resistance assay, or via a photometric measurement. Examples of reporter genes which may be mentioned are genes for resistance to antibiotics or herbicides, hydrolase genes, fluorescence protein genes, bioluminescence genes, genes for the sugar or nucleotide metabolism, or biosynthetic genes such as the Ura3 gene, the Ilv2 gene, the luciferase gene, the β-galactosidase gene, the gfp gene, the 2-deoxyglucose-6-phosphate phosphatase gene, the β-glucuronidase gene, the β-lactamase gene, the neomycin phosphotransferase gene, the hygromycinphosphotransferase gene or the BASTA (=gluphosinate resistance) gene. The transcription activity, and thus gene expression, can be measured and quantified readily owing to these genes. They allow locations of the genome to be identified which differ with regard to their productivity.

In a further advantageous embodiment, the nucleic acid sequence according to the invention may also be introduced into an organism on its own.

If it is intended to introduce further genes into the organism, besides the nucleic acid sequence according to the invention, then all of these genes together with the reporter gene can be introduced into the organism in a single vector, or each individual-gene together with the reporter gene can be introduced into in each case one vector, it being possible for the various vectors to be introduced simultaneously or successively.

The host organism advantageously comprises at least one copy of the nucleic acid according to the invention and/or of the nucleic acid construct according to the invention.

In principle, the nucleic acid according to the invention, the nucleic acid construct or the vector can be introduced into organisms, for example plants, by all methods known to the skilled worker.

For microorganisms, the skilled worker can find suitable methods in the textbooks by Sambrook, J. et al. (1989) Molecular cloning: A laboratory manual, Cold Spring Harbor Laboratory Press, by F. M. Ausubel et al. (1994) Current protocols in molecular biology, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Harbor Laboratory Press or Guthrie et al. Guide to Yeast Genetics and Molecular Biology, Methods in Enzymology, 1994, Academic Press.

The transfer of foreign genes into the genome of a plant is termed transformation. The methods which have been described for the transformation and regeneration of plants from plant tissues or plant cells are used for transient or stable transformation. Suitable methods are protoplast transformation by polyethylene glycol-induced DNA uptake, the use of a gene gun, electroporation, the incubation of dry embryos in DNA-containing solution, microinjection, and the agrobacterium-mediated gene transfer. The methods mentioned are described, for example, in B. Jenes et al., Techniques for Gene Transfer, in: Transgenic Plants, Vol. 1, Engineering and Utilization, edited by S. D. Kung and R. Wu, Academic Press (1993) 128-143 and in Potrykus Annu. Rev. Plant Physiol. Plant Molec. Biol. 42 (1991) 205-225). The construct to be expressed is preferably cloned into a vector which is capable of transforming Agrobacterium tumefaciens, for example pBin19 (Bevan et al., Nucl. Acids Res. 12 (1984) 8711). The transformation of plants with Agrobacterium tumefaciens is described, for example, by Höfgen and Willmitzer in Nucl. Acid Res. (1988) 16, 9877.

Agrobacteria transformed with an expression vector according to the invention can also be used in a known manner for the transformation of plants, such as laboratory plants such as Arabidopsis or crop plants, in particular oil-containing crop plants such as soybean, peanut, castor-oil plant, sunflower, maize, cotton, flax, oilseed rape, coconut, oil palm, safflower (Carthamus tinctorius) or cacao bean, for example by bathing scarified leaves, leaf segments, hypocotyl segments or roots in an agrobacterial solution and subsequently growing them in suitable media.

The genetically modified plant cells can be regenerated via all of the methods known to the skilled worker. Such methods can be found in the abovementioned publications by S. D. Kung and R. Wu, Potrykus or Höfgen and Willmitzer.

Suitable organisms or host organisms for the nucleic acids according to the invention, the nucleic acid constructs or the vectors are, in principle, all organisms which are capable of synthesizing fatty acids, especially unsaturated fatty acids, and which are suitable for the expression of recombinant genes. Plants which may be mentioned by way of example are Arabidopsis, Asteraceae such as Calendula, Punicaceae such as Punica granatum or crop plants such as soybean, peanut, castor-oil plant, sunflower, maize, cotton, flax, oilseed rape, coconut, oil palm, safflower (Carthamus tinctorius) or cacao bean, microorganisms such as fungi, for example the genus Mortierella, Saprolegnia or Pythium, bacteria such as the genus Escherichia, yeasts such as the genus Saccharomyces, algae or protozoans such as dinoflagellates such as Crypthecodinium. Preferred organisms are those which are capable of naturally synthesizing oils in larger quantities like fungi such as Mortierella alpina, Pythium insidiosum or plants such as soybean, oilseed rape, coconut, oil palm, safflower, castor-oil plant, Calendula, peanut, cacao bean or sunflower, or yeasts such as Saccharomyces cerevisiae, with soybean, oilseed rape, sunflower, safflower, flax, Calendula or Saccharomyces cerevisiae being especially preferred. In principle, transgenic animals, for example C. elegans, may also be used as host organisms.

A further embodiment in accordance with the invention are transgenic plants as described above comprising a functional or nonfunctional nucleic acid or a functional or nonfunctional nucleic acid construct. These transgenic plants may also comprise a vector comprising a functional or nonfunctional nucleic acid according to the invention or a functional or nonfunctional nucleic acid construct. In contrast to functional, nonfunctional is understood as meaning that enzymatically active protein is no longer synthesized. Moreover, nonfunctional nucleic acids or nucleic acid constructs are also understood as meaning what is known as antisense DNA, which leads to transgenic plants with a reduced enzymatic activity or none at all. As a rule, the enzymatic activity is reduced by 5 to 100%, preferably 10 to 90%, especially preferably 20 to 80%, very especially preferably 30 to 70%. Oils and/or triglycerides with an increased content of saturated fatty acids, or saturated fatty acids, can be synthesized with the aid of the antisense technique, specifically when the nucleic acid sequence according to the invention is combined with other fatty acid synthesis genes in the antisense DNA. Transgenic plants are understood as meaning single plant cells and their cultures on solid media or in liquid culture, plant parts and intact plants.

For the purposes of the invention, the term transgenic is understood as meaning that the nucleic acids used in the method, or the nucleic acid constructs according to the invention used in the method, are not at their natural position in the genome of an organism; in this context, the nucleic acids can be expressed homologously or heterologously. However, the term transgenic also means that the nucleic acids or expression cassettes are at their natural position in the genome of an organism, but that the sequence has been modified with respect to the natural sequence and/or that the regulatory sequences of the natural sequences have been modified. Preferably, the term transgenic is understood as meaning the expression of the nucleic acids according to the invention at a position in the genome which is not their natural position; that is to say, the nucleic acids are expressed homologously or, preferably, heterologously. Preferred transgenic organisms are the abovementioned transgenic plants, preferably oil crop plants.

The subjects of the invention therefore also include the use of the nucleic acid sequence according to the invention or the nucleic acid construct according to the invention in their functional or nonfunctional forms for generating transgenic plants.

The invention furthermore therefore also relates to a process for the production of oils or triglycerides with an increased content of saturated fatty acids, which comprises the following process steps:
a) introducing at least one nonfunctional nucleic acid sequence as claimed in claim 1, at least one nonfunctional nucleic acid construct as claimed in claim 4 or a vector comprising such nonfunctional nucleic acids or nucleic acid constructs into an oil-producing organism;
b) culturing this organism and
c) isolating the oil present in the organism, or triglycerides present in the organism.

The saturated fatty acids can be liberated from the oils and/or triglycerides thus obtained by methods known to the skilled worker. Liberation is effected by what is known as acid or alkaline hydrolysis of the ester bonds. Alkaline hydrolysis, for example with NaOH or KOH is preferred. If it is intended to prepare the alkyl esters, such as the methyl or ethyl esters, of the saturated or, as written further above, the unsaturated fatty acids, the hydrolysis can advantageously be carried out with the corresponding alkoxides.

Organisms which are preferably employed for the processes according to the invention for the production of oils and/or triglycerides with an increased content of unsaturated or saturated fatty acids and, if appropriate, their subsequent liberation via hydrolysis, are plants, especially preferably oil crop plants, or microorganisms.

The invention furthermore relates to nucleic acids encoding a protein which converts a fatty acid of the structure I, 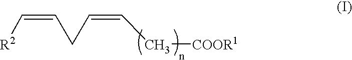
which has two double bonds separated from each other by a methylene group, to a triunsaturated fatty acid of the structure II 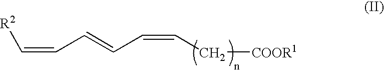
in which the three double bonds of the fatty acid are conjugated and in which the substituents and variables in the compounds of the structure I and II have the following meanings:
R¹=hydrogen, substituted or unsubstituted, unsaturated or saturated, branched or unbranched C₁-C₁₀-alkyl-, 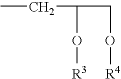
R²=substituted or unsubstituted, unsaturated or saturated C₁-C₉-alkyl-,
R³ and R⁴ independently of one another, hydrogen, substituted or unsubstituted, saturated or unsaturated, branched or unbranched C₁-C₂₂-alkylcarbonyl- or phospho,
n=1 to 14, preferably 1 to 8, especially preferably 4 to 7, very especially preferably 7.

In the compounds of the formula I and II, R¹ is hydrogen, substituted or unsubstituted, unsaturated or saturated, branched or unbranched C₁-C₁₀-alkyl-, or
Alkyl radicals which may be mentioned are substituted or unsubstituted, branched or unbranched C₁-C₁₀-alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl or n-decyl.

Preferred radicals for R¹ are hydrogen and

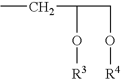

In the compounds of the formula I and II, R² is substituted or unsubstituted, unsaturated or saturated C₁-C₉-alkyl-.

Alkyl radicals which may be mentioned are substituted or unsubstituted, branched or unbranched C₁-C₉-alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl or n-nonyl. C₁-C₅-alkyl is preferred, C₄- and C₅-alkyl is especially preferred, the butyl radical is very especially preferred.

R³ and R⁴ independently of one another are hydrogen, substituted or unsubstituted, saturated or unsaturated, branched or unbranched C₁-C₂₂-alkylcarbonyl- or phospho-.

C₁-C₂₂-Alkylcarbonyl being understood as meaning radicals such as methylcarbonyl, ethylcarbonyl, n-propylcarbonyl, 1-methylethyl-carbonyl, n-butylcarbonyl, 1-methylpropylcarbonyl, 2-methyl-propylcarbonyl, 1,1-dimethylethylcarbonyl, n-pentylcarbonyl, 1-methylbutylcarbonyl, 2-methylbutylcarbonyl, 3-methylbutyl-carbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethyl-butylcarbonyl, 1,2-dimethylbutylcarbonyl, 1,3-dimethylbutyl-carbonyl, 2,2-dimethylbutylcarbonyl, 2,3-dimethylbutylcarbonyl, 3,3-dimethylbutylcarbonyl, 1-ethylbutylcarbonyl, 2-ethylbutyl-carbonyl, 1,1,2-trimethylpropylcarbonyl, 1,2,2-trimethylpropyl-carbonyl, 1-ethyl-1-methylpropylcarbonyl and 1-ethyl-2-methyl-propylcarbonyl, heptylcarbonyl, nonylcarbonyl, decylcarbonyl, undecylcarbonyl, n-dodecylcarbonyl, n-tridecylcarbonyl, n-tetradecylcarbonyl, n-pentadecylcarbonyl, n-hexadecylcarbonyl, n-heptadecylcarbonyl, n-octadecylcarbonyl, n-nonadecylcarbonyl or n-eicosylcarbonyl.

Substituents which are preferred for R³ and R⁴ are saturated or unsaturated C₁₆-C₂₂-alkylcarbonyl.

Substituents of the abovementioned radicals which may be mentioned by way of example are halogen such as fluorine or chlorine, alkyl or hydroxyl.

The reaction with the protein or enzyme according to the invention introduces a double bond into the fatty acid and shifts a double bond, so that the three double bonds which participate in the reaction are conjugated. Furthermore, one double bond is isomerized (from cis to trans).

The enzyme (=punicic acid desaturase) advantageously catalyzes the reaction from linoleic acid (18:2, 9Z,12Z) to punicic acid (18:3, 9Z,11E,13Z). The enzyme introduces a cis double bond at position C₁₃ and causes the specific shift of a cis double bond in position C₁₂ to a trans double bond in position C₁₁, the isomerization taking place in a regiospecific manner.

The reaction probably first proceeds via a 1,4 elimination and a subsequent 11,14 desaturation. Another suitable substrate is γ-linolenic acid (18:3, 6Z,9Z,12Z), which is then converted into the corresponding conjutetraene (18:4, 6Z,9Z,11E,13Z).

Further suitable as substrate are oleic acid (18:1, 9Z) and vaccenic acid (18:1, 11Z), which is then converted into conjugated linoleic acid. Advantageously, the reaction gives preferentially the 9_{cis},11ₜᵣₐₙₛ isomer.

The invention furthermore relates to a process for the production of fatty acid mixtures with an increased content of unsaturated fatty acids, which comprises introducing at least one above-described nucleic acid sequence according to the invention or at least one nucleic acid construct according to the invention into a preferably oil-producing organism, culturing this organism, and isolating the oil and/or triglyceride present in the organism and liberating the fatty acids present in the oil and/or triglyceride.

The subjects of the invention also include a process for the production of oils and/or triglycerides with an increased content of unsaturated fatty acids, which comprises introducing at least one above-described nucleic acid sequence according to the invention or at least one nucleic acid construct according to the invention into an oil-producing organism, culturing this organism and isolating the oil present in the organism.

Both processes advantageously make possible the synthesis of fatty acid mixtures or triglycerides with an increased content punicic acid.

The invention furthermore relates to a process for the production of saturated fatty acids, which comprises introducing at least one nonfunctional abovementioned nucleic acid sequence according to the invention or at least one nonfunctional nucleic acid construct according to the invention into an oil-producing organism, culturing this organism, isolating the oil present in the organism and liberating the fatty acids present in the oil, and a process for the production of triglycerides with an increased content of saturated fatty acids, which comprises introducing at least one nonfunctional abovementioned nucleic acid sequence according to the invention or at least one nonfunctional nucleic acid construct according to the invention into an oil-producing organism, culturing this organism and isolating the oil present in the organism. Both processes rely on what is known as antisense technology (see above).

Examples of organisms for the abovementioned processes which may be mentioned by way of example are plants such as Arabidopsis, soybean, peanut, castor-oil plant, sunflower, maize, cotton, flax, oilseed rape, coconut, oil palm, safflower (Carthamus tinctorius) or cacao bean, microorganisms like fungi [lacuna] Mortierella, Saprolegnia or Pythium, bacteria such as the genus Escherichia, yeasts such as the genus Saccharomyces, algae or protozoa such as dinoflagellates such as Crypthecodinium. Preferred organisms are those which are capable of naturally synthesizing larger amounts of oils, like fungi such as Mortierella alpina, Pythium insidiosum or plants such as soybean, oilseed rape, coconut, oil palm, safflower, castor-oil plant, Calendula, Punica, peanut, cacao bean or sunflower, or yeasts such as Saccharomyces cerevisiae; soybean, oilseed rape, sunflower, Calendula, Punica or Saccharomyces cerevisiae being especially preferred.

Depending on the host organism, the organisms used in the processes are grown or cultured in a manner with which the skilled worker is familiar. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as iron salts, manganese salts and magnesium salts and, if appropriate, vitamins, at temperatures of between 0° C. and 100° C., preferably between 10° C. and 60° C., while passing in oxygen. The pH of the liquid medium may be kept constant, that is to say regulated during the culturing, or not. They can be cultured in the form of a batch process, a semi-batch process or a continuous process. Nutrients can be provided at the beginning of the fermentation or resupplied semicontinuously or continuously.

After the transformation, plants are first regenerated as described above and subsequently grown or sown as usual.

After the organisms have been cultured, the lipids can be obtained in the customary manner. To this end, the organisms can first be harvested and then disrupted, or they may be used directly. The lipids are advantageously extracted with suitable solvents such as apolar solvents such as hexane or ethanol, isopropanol or mixtures such as hexane/isopropanol, phenol/chloroform/isoamyl alcohol at temperatures between 0° C. and 80° C., preferably between 20° C. and 50° C. As a rule, the biomass is extracted with an excess of solvent, for example an excess of solvent to biomass of 1:4. The solvent is subsequently removed, for example by distillation. Extraction may also be effected with supercritical CO₂. After extraction, the remainder of the biomass can be removed for example by filtration.

The crude oil thus obtained can subsequently be purified further, for example by removing the turbidity via treatment with polar solvent such as acetone or chloroform and subsequently filtering or centrifuging the mixture. Further purification via columns is also possible.

To obtain the free fatty acids from the triglycerides, the latter are saponified in the customary manner.

The invention therefore furthermore relates to fatty acid mixtures with an increased content of unsaturated fatty acids and to oils and/or triglyceries with an increased content of unsaturated fatty acids, which have been prepared by the abovementioned processes, and to their use for the preparation of foodstuffs, animal feeds, cosmetics or pharmaceuticals. To this end, they are added to the foodstuffs, the animal feed, the cosmetics or the pharmaceuticals in customary amounts.

The invention is illustrated hereinbelow with reference to examples:

### EXAMPLES

A cDNA was cloned from Punica granatum mRNA via RT-PCR and RACE techniques. When this cDNA is expressed in yeast, linoleic acid is converted into the octadecaconjutriene punicic acid (9Z,11E,13Z). As far as we know, this is the first description of a punicic acid desaturase. The enzyme causes a regiospecific shift of a cis double bond in position C₁₂ to a trans double bond in position C₁₁ and introduces a new cis double bond at position C₁₃. A cDNA which encodes a functional Δ-12-desaturase was also cloned.

Transgenic yeasts and plants with an increased expression of the punicic acid desaturase cDNA contain punicic acid in their lipids. The punicic acid synthesis can be increased by additionally expressing a Δ-12-desaturase, which leads to an increased content of linoleic acid which, in turn, constitutes the substrate for punicic acid desaturase.

### Example 1

### RNA Isolation from Punica granatum seeds

RNA was isolated from Punica granatum seeds by the method described in Fritsche et al. (FEBS Letters, 462, 1999, 249-253). The following steps were modified: after centrifugation for 18 hours and washing of the pellets with 70% strength ethanol, the subsequent extraction was carried out not for 15 minutes, but for 1 hour at 65° C. in the waterbath, with vortexing every 10 minutes. The mRNA was isolated from 3 mg of total RNA using Promega's Poly-Attract kit following the manufacturer's instructions. ss-cDNA was generated from 1 μg of mRNA using oligo(dT) primer, Superscript II from Gibco-BRL following the manufacturer's instructions. This ss-cDNA was employed as template in a polymerase chain reaction (PCR).

### Example 2

### Isolation and Cloning of the Punica granatum Punicic Acid Desaturate and Δ-12-desaturase

In order to isolate DNA sequences from Punica granatum which encode a punicic acid desaturase and a Δ-12-desaturase, various degenerate oligonucleotide primers were derived from amino acid sequences of the conserved histidine boxes of various Δ-12-desaturases.
Primer A: 5′-TGG GTI AWH GCH CAY GAR TGB GG-3′ Forward primer, derived from the amino acid sequence W V I A H E C
Primer B: 5′-GGC ATI GTI GAR AAS ARR TGR TGV AGY MAC-3′ Reverse primer, derived from the amino acid sequence V T/A H H L F S T I
Primer C: 5′-CCD TAY TTC TCI TGG AAR WWH AGY CAY CG-3′ Forward primer, derived from the amino acid sequence P Y F S W K Y/I S H R
Primer D: 5′-CCA RTY CCA YTC IGW BGA RTC RTA RTG-3′ Reverse primer, derived from the amino acid sequence H Y D S S/T E W D/N W

The letters in primers A, B, C and D have the following meaning:
R=A/G
Y=C/T
W=A/T
H=A/C/T
B=C/G/T
D=A/G/T
I=inositol

DNA fragments were amplified in a PCR with Punica single-strand cDNA (prepared as described in Example 1) as template, using the primer combinations A/B and A/D. The Biozyme Tfl polymerase was employed for the amplification.

The PCR reaction mix was composed as follows:

| | |
|---|---|
| dNTP mix (10 mM) | 0.5 μl |
| Forward primer (10 μM) | 2.5 μl |
| Reverse primer (10 μM) | 2.5 μl |
| Template (ss-DNA) | 1.0 μl |
| 20 × Tfl buffer | 1.25 μl |
| MgCl2 (25 mM) | 2.5 μl |
| Tfl polymerase (1 U/μl) | 0.25 μl |
| Water | 14.5 μl |
| Total volume | 25.0 μl |

The following PCR program was used:

| | | |
|---|---|---|
| 1. | 2 min | 94° C. |
| 2. | 30 sec | 94° C. |
| 3. | 45 sec | 50° C. (annealing temperature) |
| 4. | 1 min | 72° C. |
| 5. | 10 × 2. to 4. | |
| 6. | 0 sec | 94° C. |
| 7. | 45 sec | 50° C. |
| 8. | 1 min | 72° C., time increment 5 sec per cycle |
| 9. | 20 × 5. to 7. | |
| 10. | 2 min | 72° C. |

The primer combinations A/B and A/D gave PCR fragments. They were excised from a preparative agarose gel, eluted with GFX™ PCR DNA and Gel Band Purification kit from Amersham Pharmacia Biotech and cloned into the pGEM-T vector system (Promega) following the manufacturer's instructions. Clones which differed from each other were sequenced using M13 primers.

The sequences of these approximately 570 bp PCR products can be seen from SEQ ID NO: 1 and SEQ ID NO: 4.

### Example 3

### Obtaining and Sequencing Complete cDNA Clones

To obtain full-length clones, the fragments SEQ ID NO: 1 and SEQ ID NO: 4 were elongated by 5′- and 3′-RACE (rapid amplification of cDNA ends). Starting from 1 μg of mRNA (isolated as described in Example 1), a “Marathon cDNA library” was constructed with the “Marathon cDNA amplification kit” from CLONTECH (Heidelberg) following the manufacturer's instructions.

The 5′- and 3′-RACE PCR was carried out using the Advantage cDNA PCR kit from Clontech following the manufacturer's instructions and using the following gene-specific RACE primers:
Primer E: 5′-ACG GAA CGA GGA GCG CTG AGT G-3′ Specific primer for the 5′-RACE of punicic acid desaturase
Primer F: 5′-CTG ATC GTG AAC GCA TTC CTG G-3′ Specific primer for the 3′-RACE of punicic acid desaturase
Primer G: 5′-GGG ACG AGG AGC GAT GTG TGG AG-3′ Specific primer for the 5′-RACE of Δ-12-desaturase
Primer H: 5′-AGT CCT CAT ATT AAA TGC ATT CGT GG-3′ Specific primer for the 3′-RACE of Δ-12-desaturase

The PCR reaction was composed as follows:

| | |
|---|---|
| dNTP mix (10 mM) | 1.0 μl |
| RACE primer (10 μM) | 1.0 μl |
| Adapter primer (10 μM) | 1.0 μl |
| Template | 1.0 μl |

(Marathon cDNA library diluted 1:50)

| | |
|---|---|
| 10 × buffer | 5.0 μl |
| Polymerase (1 U/μl) | 1.0 μl |
| Water | 38.5 μl |
| Total volume | 50.0 μl |

The RACE PCR was carried out with the following program:

| | | |
|---|---|---|
| 1. | 1 min | 94° C. |
| 2. | 30 sec | 94° C. |
| 3. | 3 min | 68° C. |
| 4. | 10 × 2.-3. | |
| 5. | 30 sec | 94° C. |
| 6. | 30 sec | 65° C. |
| 7. | 3 min | 68° C. |
| 8. | 25 × 4.-6. | |
| 9. | 5 min | 68° C. |

The DNA fragments obtained were excised from a preparative agarose gel as described in Example 2, eluted with the GFX™ PCR DNA and Gel Band Purification kit from Amersham Pharmacia Biotech, cloned into the pGEM-T vector system (Promega) following the manufacturer's instructions and sequenced. The 5′-RACE products extended beyond the start codon into the 5′-untranslated region (5′-UTR) and the 3′-RACE products extended beyond the stop codon into the 3′-UTR. The assembled DNA sequences of punicic acid desaturases and Δ-12-desaturase are shown in SEQ ID NO: 2; SEQ ID NO: 5 and SEQ ID NO: 7. The sequences encompass the coding region and a segment of the 5′-UTR and the 3′-UTR.

The coding region of SEQ ID NO: 2 (punicic acid desaturase, PuFADX) extends from nucleotide 131 to 1252, that of SEQ ID NO: 5 (Δ-12-desaturase) from nucleotide 94 to 1254 and that of SEQ ID NO: 7 (punicic acid desaturase, PuFADX2) from nucleotide 1 to 1188.

To obtain contiguous full-length clones, the coding regions of punicic acid desaturases and of Δ-12-desaturase were amplified and cloned. This was done using the Expand High Fidelity PCR system (Roche Diagnostics) and the primers I and J for punicic acid desaturases or the primers K and L for delta-12-desaturase and with Punica cDNA as template.
Primer I: 5′-AAG CTT ATG GGA GCT GAT GGA ACA ATG TCT C-3′ Forward primer (with HindIII cleavage site)
Primer J: 5′-GGA TCC ATT CAG AAC TTG CTC TTG AAC CAT AG-3′ Reverse primer (with BamHI cleavage site)
Primer K: 5′-GTC GAC ATG GGA GCC GGT GGA AGA ATG AC-3′ Forward primer (with SalI cleavage site)
Primer L: 5′-AAG CTT TGA TCA GAG GTT CTT CTT GTA CCA G-3′ Reverse primer (with HindIII cleavage site)

The PCR reactions were composed as follows:

| | |
|---|---|
| dNTP mix (10 mM) | 1.0 μl |
| Forward primer (10 μM) | 4.0 μl |
| Reverse primer (10 μM) | 4.0 μl |
| Template | 3.0 μl |

(Marathon-cDNA bank library diluted 1:50)

| | |
|---|---|
| 10 × buffer 2 | 5.0 μl |
| Polymerase (3.5 U/μl) | 0.5 μl |
| Water | 32.5 μl |
| Total volume | 50.0 μl |

The PCR was carried out with the following program:

| | | |
|---|---|---|
| 1. | 2 min | 94° C. |
| 2. | 30 sec | 94° C. |
| 3. | 30 sec | 58° C. |
| 4. | 1 min | 72° C. |
| 5. | 10 × 2.-4. | |
| 6. | 30 sec | 94° C. |
| 7. | 30 sec | 58° C. |
| 8. | 1 min | 72° C., time increment 5 sec per cycle |
| 9. | 15 × 5.-7. | |
| 10. | 5 min | 72° C. |

The 1.2 kb PCR products were cloned into the vector pGEM-T (Promega, Mannheim) and transformed into E. coli XL1 blue cells. The insert DNA was sequenced (both strands) using a 373 DNA sequencer (Applied Biosystems) and was identical in each case with the coding regions of the punicic acid desaturases (SEQ ID NO: 2 and SEQ ID NO: 7) and of the Δ-12-desaturase (SEQ ID NO: 5).

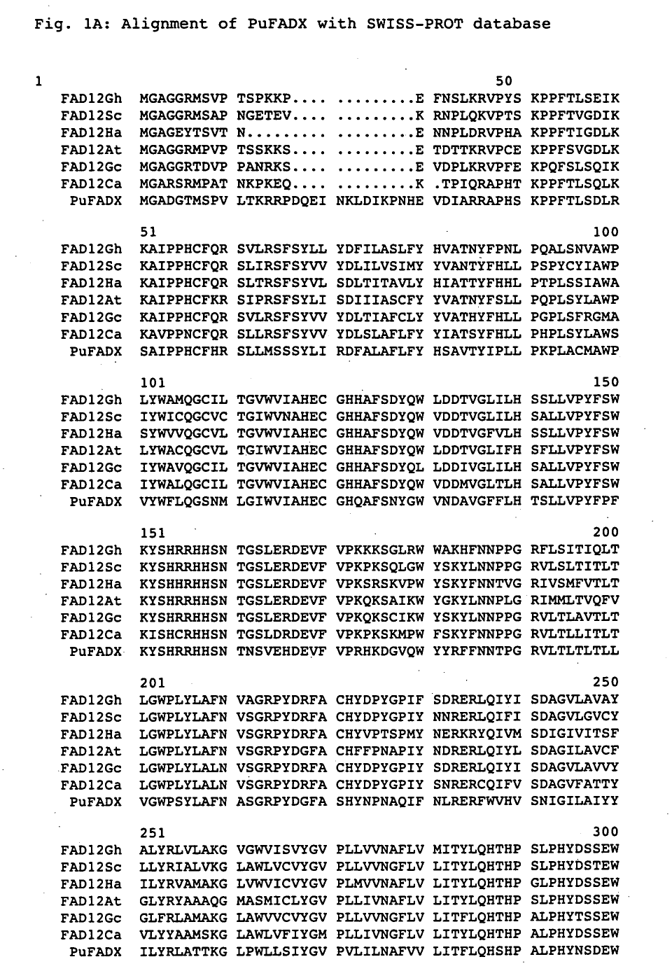

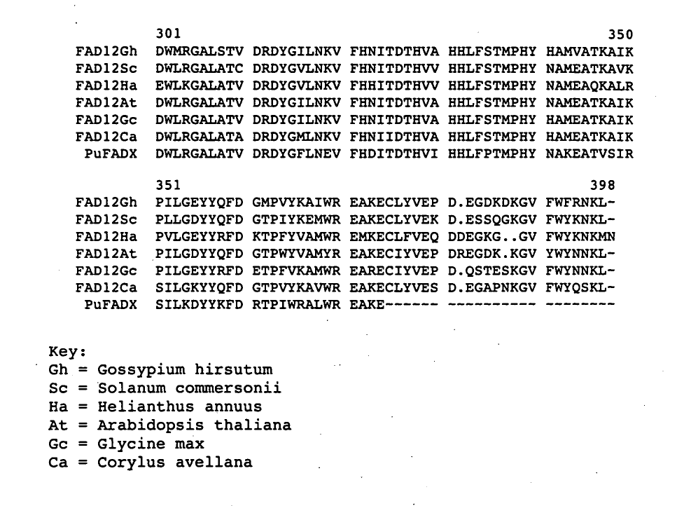

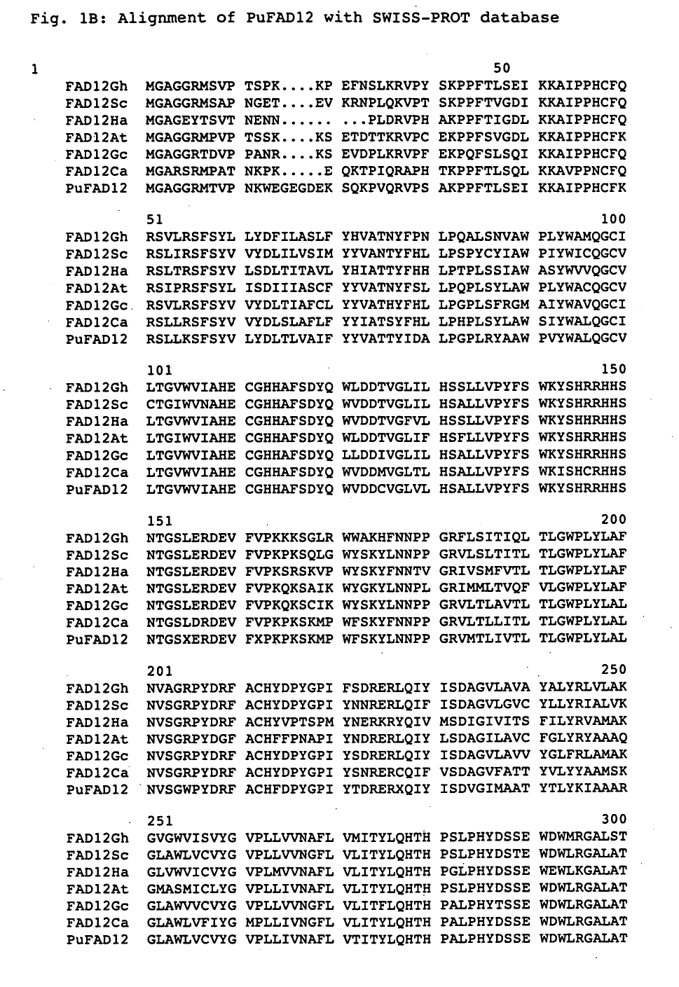

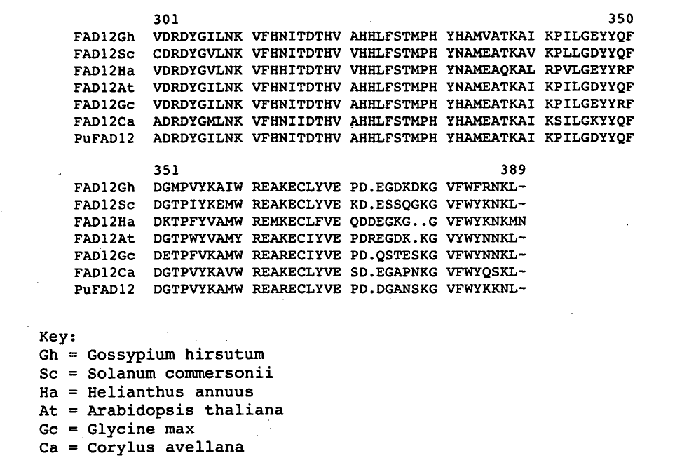

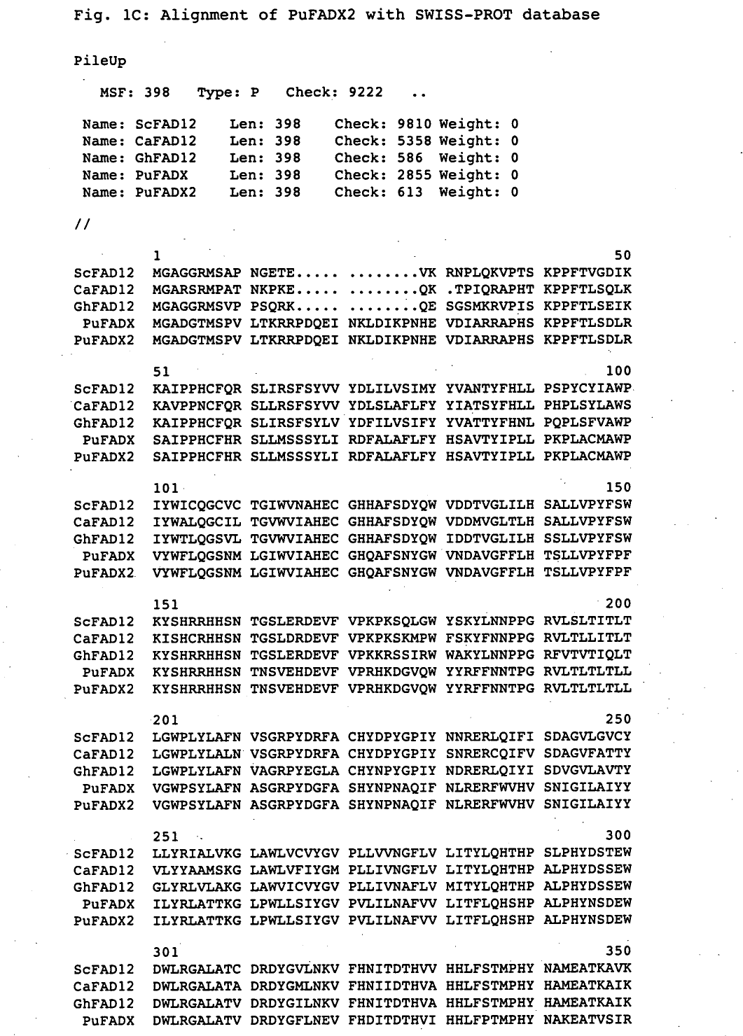

An alignment of the derived amino acid sequence of PuFADX (SEQ ID NO: 2) with annotated protein sequences of the SWISS-PROT and SP-TREMBL databases revealed the highest homology with a Solanum commersonii Δ-12-desaturase (60% identical amino acids), a Corylus avellana Δ-12-desaturase (60% identical amino acids) and an Ω-6 fatty acid desaturase from cotton (58% identical amino acids) over the entire coding region. FIG. 1A shows an alignment of the PuFADX amino acid sequences with Gossypium hirsutum, Solanum commersonii, Helianthus annuus, Arabidopsis thaliana, Glycine max and Corylus avellana Δ-12-desaturases.

An alignment of the derived amino acid sequence of PuFAD12 (SEQ ID NO: 5) with annotated protein sequences from SWISS-PROT and SP-TREMBL databases revealed the highest homologies with Δ-12-desaturases from Sesamum indicum (78% identical amino acids), from Corylus avellana (78% identical amino acids) and from Solanum commersonii (77% identical amino acids). FIG. 1B shows an alignment of the PuFAD12 amino acid sequences with Gossypium hirsutum, Solanum commersonii, Helianthus annus, Arabidopsis thaliana, Glycine max and Corylus avellana Δ-12-desaturases.

An alignment of the derived amino acid sequence of PuFADX2 (SEQ ID NO: 7) with annotated protein sequences of the SWISS-PROT and SP-TREMBL databases revealed the highest homology with a Gossypium hirsutum Δ-12-desaturase (59% identical amino acids), a Corylus avellana Δ-12-desaturase (58% identical amino acids) and a Solanum commersonii Δ-12-desaturase (58% identical amino acids). FIG. 1C shows an alignment of PuFADX2 amino acid sequences (=SEQ ID NO: 7) with Gossypium hirsutum, Solanum commersonii and Corylus avellana Δ-12-desaturases and with PuFADX (=SEQ ID NO: 2).

### Example 4

### Expression of Punicic Acid Desaturase and Δ-12-desaturase in Yeast

In order to demonstrate the functionality of PuFADX, the coding region of the cDNA was, in a first approach, cloned into a yeast expression vector and expressed in S. cerevisiae. The punicic acid desaturase produced in the yeast was intended to convert added linoleic acid into punicic acid. The latter, in turn, was to be detected in hydrolyzed and transmethylated lipid extracts via GC and GC/MS in the form of the methyl ester.

In a second approach, the Δ-12-desaturase PuFAD12 was expressed in yeast, in addition to PuFADX, so that the yeast cells endogenously produced linoleic acid which then, in turn, can be converted into punicic acid owing to the PuFADX activity. The punicic acid, in turn, was to be detected via GC and GC/MS.

All of the yeast solid and liquid media were prepared following protocols of Ausubel et al. (Current Protocols in Molecular Biology, John Wiley & Sons, New York, 1995).

The PuFADX cDNA was excised from the vector pGEM-T via restriction digest with HindIII/BamHI, cloned into the HindIII/BamHI-cut shuttle vector pYES2 (Invitrogen, Carlsbad, USA), and the resulting vector pYES2-PuFADX was transformed into E. coli XL1 blue. pYES2-PuFADX was transformed into S. cerevisiae INCSc1 (Invitrogen, Carlsbad, USA) with the aid of the LiAc method, the expression of the PuFADX-cDNA being under the control of the GAL1 promoter.

To allow the expression in yeast of PuFAD12 in addition to PuFADX in the second approach, the PuFAD12-cDNA was first excised from vector pGEM-T via restriction digest with SalI/HindIII, cloned into the SalI/HindIII-cut shuttle vector pESC-Leu (Stratagene), and the resulting vector pEST-Leu-PuFADX was transformed into E. coli XL1 blue. pYES2-PuFAD12 was transformed into S. cerevisiae INCSc1 (Invitrogen, Carlsbad, USA) with the aid of the LiAc method, the expression of the PuFAD12-cDNA being under the control of the GAL1 promoter.

PuFADX was expressed in S. cerevisiae INVSc1 by the modified method of Avery et al. (Appl. Environ. Microbiol., 62, 1996: 3960-3966) and Girke et al. (The Plant Journal, 5, 1998: 39-48). To produce a starter culture, 20 ml of SD medium with glucose and histidine-free amino acid solution was inoculated with a single yeast colony and incubated overnight at 30° C. and 140 rpm. The cell culture was washed twice by spinning down and resuspending the pellet in SD medium without supplements and without sugars. A main culture was inoculated to an OD₆₀₀ of 0.1 to 0.3 with the washed cells. The main culture was grown for 72 hours at 30° C. in 25 ml of SD medium with 2% (w/v) galactose, histidine-free amino acid solution, 0.02% linoleic acid (2% strength stock solution in 5% Tergitol NP40), 10% Tergitol NP40. The main culture was harvested by centrifugation. The cell pellet was frozen at −20° C. and subsequently lyophilized for approximately 18 hours.

PuFAD12 was expressed analogously, with the following differences: the culture was not selected for hisitidine prototrophism, but for leucine prototrophism; the volume of the main culture was 50 ml, and the fermentation conditions of the main cultures were 240 hours at 16° C.

To investigate the substrate specificity of punicic acid desaturase, other fatty acids were added to the yeast culture instead of linoleic acid, such as, for example, oleic acid, cis-vaccenic acid, trans-vaccenic acid, gamma-linolenic acid, alpha-linolenic acid.

PuFADX2 was expressed in yeasts in analogy to the example described above.

### Example 5

### Lipid Extraction of the Fatty Acids from Transgenic Yeast, and GC and GC/MS Analysis

The lyophilized yeast cells were extracted in 1.35 ml of methanol/toluene (2:1) and 0.5 ml of sodium methoxide solution. The cell material was broken up as finely as possible with a glass rod and then incubated with shaking for 1 hour at room temperature (room temperature=RT means approximately 23° C. in the present application).

Then, 1.8 ml of 1 M NaCl solution and 3 ml of n-heptane were added and the mixture was incubated with shaking for 10 minutes at room temperature. Following phase separation by centrifugation (10 min, 400 rpm, 4° C.), the heptane supernatant was transferred into a test tube and evaporated to dryness under nitrogen. The residue was taken up in 3 times 0.3 ml of hexane, transferred into an Eppendorf tube and again evaporated to dryness under nitrogen. The residue was taken up in 50 μl of MeCN and the sample was analyzed by GC or GC/MS.

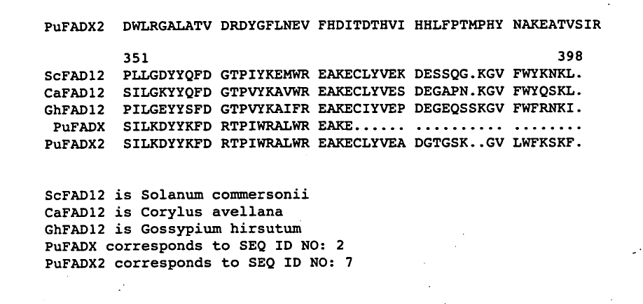

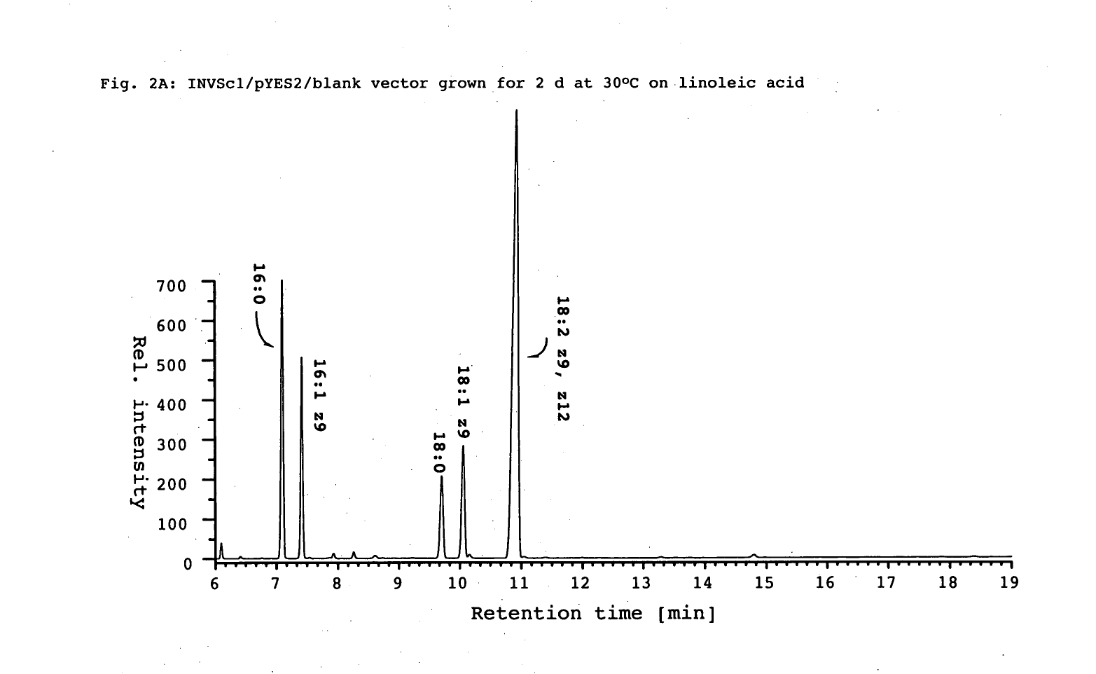

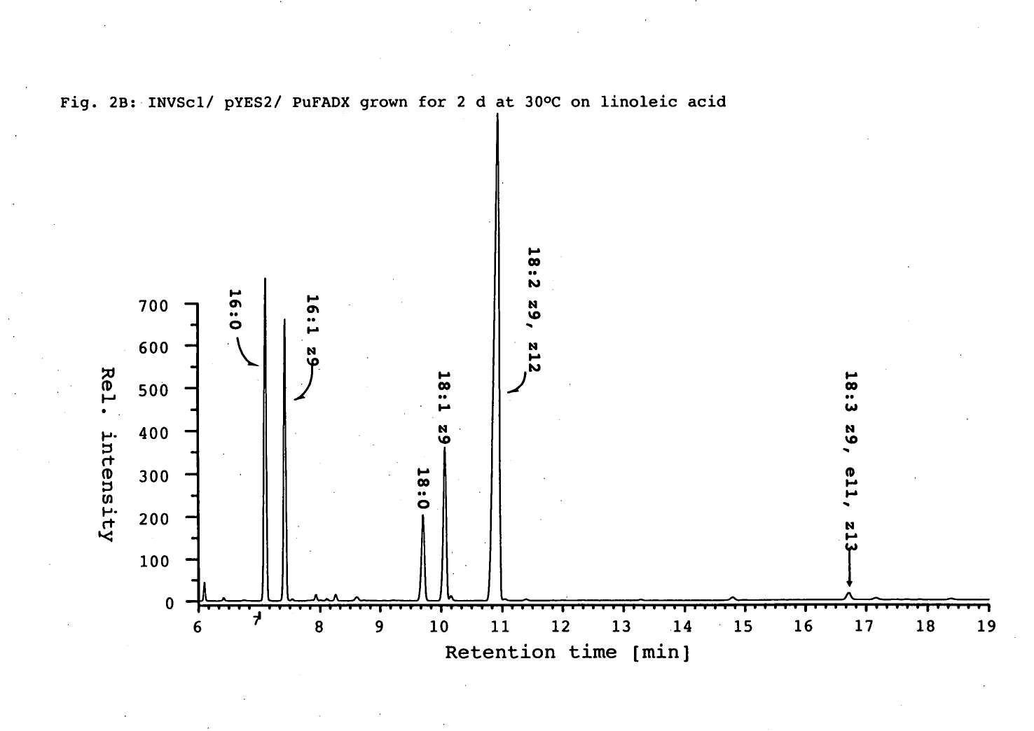

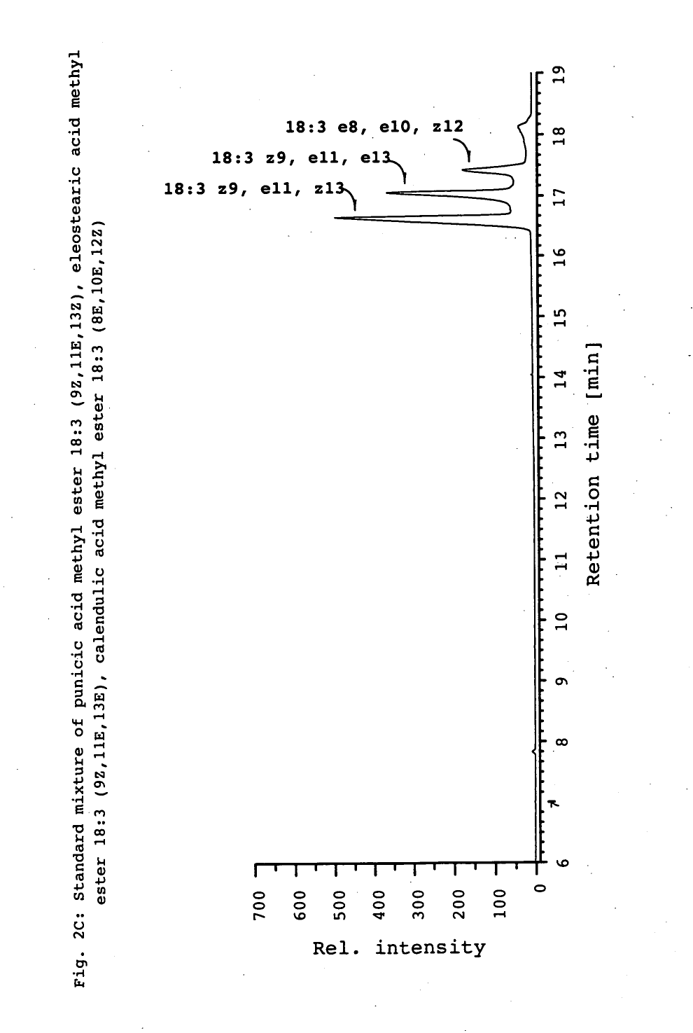

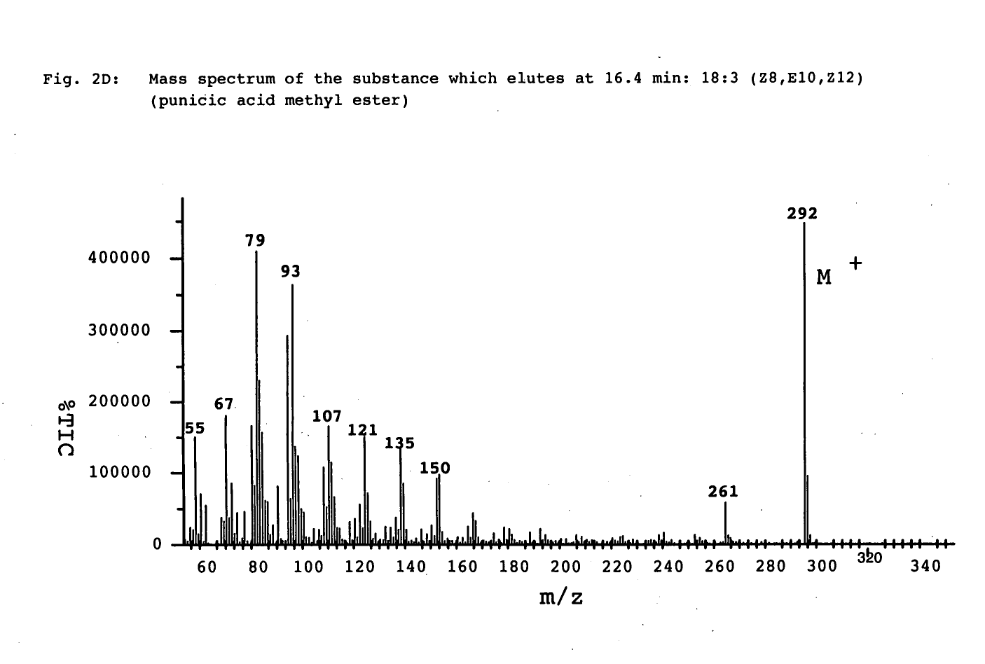

To carry out the GC analysis of the fatty acid methyl esters (FAME) 7 μl of the sample (in MeCN) were transferred into a test tube and 1 μl was injected. The GC analysis was carried out using an HP-INNOWax column (Crosslinked PEG; 30 m×0.32 mm×0.5 μm film thickness) at a flow rate of 1.5 ml/min. Helium acted as the carrier gas. The injection temperature was 220° C. The following temperature gradient was applied: 1 min 150° C., 150° C. to 200° C. (15° C./min), 200° C. to 250° C. (2° C./min), 5 min 250° C. The FAMEs were detected via a flame ionization detector (FID) at 275° C. The retention times of octadecaconjutriene FAMEs are 16.6 min for punicic acid, 17.0 min for eleostearic acid and 17.4 min for calendulic acid (FIG. 2C). The retention times of octadecaconjutetraene FAMEs are 17.0.min for 18:4 (6Z,9Z,11E,13Z) and 17.4 min for 18:4 (6Z,9Z,11E,13Z) (not shown).

FIG. 2 shows the production of punicic acid in yeast cells transformed with the Punica granatum punicic acid desaturase. FIG. 2A shows the gas chromatogram of the lipid extracts from yeast cells transformed with the blank vector pYES2. The cells were grown for 72 hours at 30° C. with 0.02% linoleic acid as described in Example 4. The gas chromatogram shows no FAMEs with a retention time of punicic acid. FIG. 2B shows the gas chromatogram of the lipid extracts from yeast cells transformed with pYES2-PuFADX. Again, the cells were grown for 72 hours at 30° C. with 0.02% linoleic acid as described in Example 4. The gas chromatogram shows a pronounced peak with a retention time of 16.6 min, which is not observed in the control batch (cf. FIG. 2A) and has the same retention time as punicic acid (cf. FIG. 2C). Yeasts which were transformed with SEQ ID NO: 7 (PuFADX2) were analyzed analogously.

To carry out the GC/MS analysis of the FAMEs, 20 μl of the sample (in MeCN) were transferred into a test tube, and 4 μl were injected. An HP-5 column (5% diphenyl/95% polydimethylsiloxane, 30 m×0.25 mm, film thickness 0.25 μm, Agilent, Waldbronn) was used in the GC analysis. Helium acted as the carrier gas (40 cm/s). The samples were measured in the EI mode, the injection temperature was 250° C. The temperature program used was: 60° C. to 110° C. (25° C./min), 1 min 110° C., 110° C. to 270° C. (10° C./min), 10 min 270° C.

When using the HP-5 column under the conditions described, various C₁₈-fatty acids have the following retention times:
18:0=15.7 min
18:1 9Z=15.6 min
18:2 9Z,12Z=15.2 min
18:3 9Z,11E,13Z=16.4 min
18:4 6Z,9Z,11E,13Z=16.25 min

FIG. 2D shows the mass spectrum of the compound which, according to GC with an HP-5 column, has a retention time of 16.4 min. The material analyzed was a lipid extract of yeast cells transformed with pYES2-PuFADX, that is to say which express punicic acid desaturase. The mass spectrum allowed the unambiguous identification of the compound as the methyl ester of an octadecaconjutriene.

The results shown in FIG. 2 demonstrate that punicic acid desaturase in yeast leads to the formation of punicic acid by conversion of linoleic acid. The detection of punicic acid from transformed yeast cells was only successful after the lipids had been hydrolyzed. No punicic acid was detected in the free fatty acids of these cells, that is to say that, in yeast, punicic acid is incorporated into lipids. Since yeast contains virtually no triacyl glycerides, it must be assumed that most of the punicic acid found had been bound in the yeast phospholipids.

FIG. 3 shows the formation of octadecaconjutetraenoic fatty acids in yeast cells which were transformed with the Punica granatum punicic acid desaturase and grown with γ-linolenic acid as described in Example 4. FIG. 3A shows the gas chromatogram of the lipids extracts from control cells transformed with the blank vector pYES2. The cells were grown for 72 hours at 30° C. with 0.02% γ-linolenic acid. The gas chromatogram shows no FAMEs with a retention time of octadecaconjutetraenoic fatty acids (17.0 min-17.4 min).

FIG. 3B shows the gas chromatogram of the lipid extracts from yeast cells transformed with pYES2-PuFADX. Again, the cells were grown for 72 hours at 30° C. with 0.02% γ-linolenic acid as described in Example 4. The gas chromatogram shows pronounced peaks with retention times of 17.0 min and 17.4 min, which are not found in the control batch (cf. FIG. 3A) and which has the same retention time as 18:4 (6Z,9Z,11E,13Z).

A GC/MS analysis of this compound extracted from transgenic yeast cells with pYES2-PuFADX which had been grown with γ-linolenic acid is shown in FIG. 3C. The mass spectrum allows the unambiguous identification of the compound as the methyl ester of an octadecaconjutetraene.

The results shown in FIG. 3 show that punicic acid desaturase converts γ-linolenic acid in yeast into octadecaconjutetraenoic fatty acids. α-Linolenic acid, in contrast, did not lead to the formation of octadecaconjutetraenes (not shown). In yeast, the abovementioned octadecaconjutetraenes are incorporated into lipids, predominantly into phospholipids.

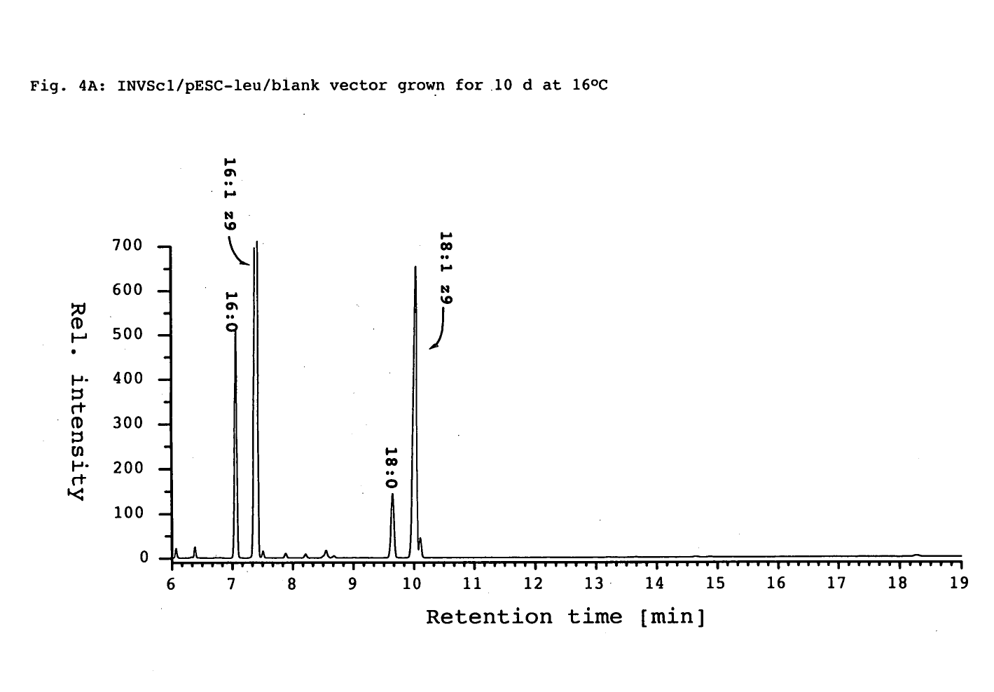

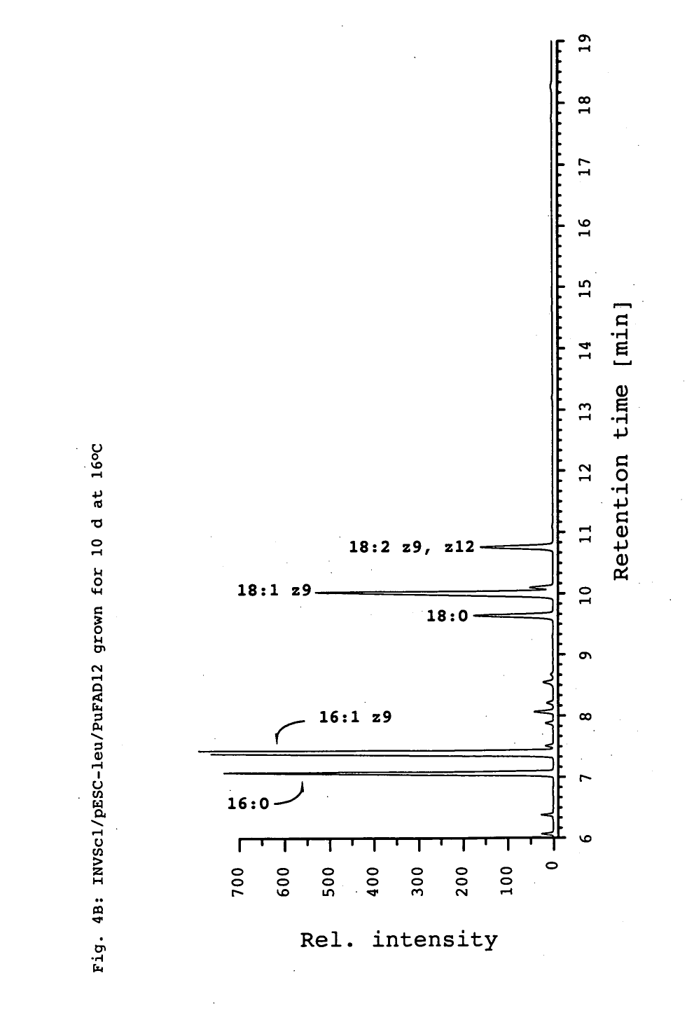

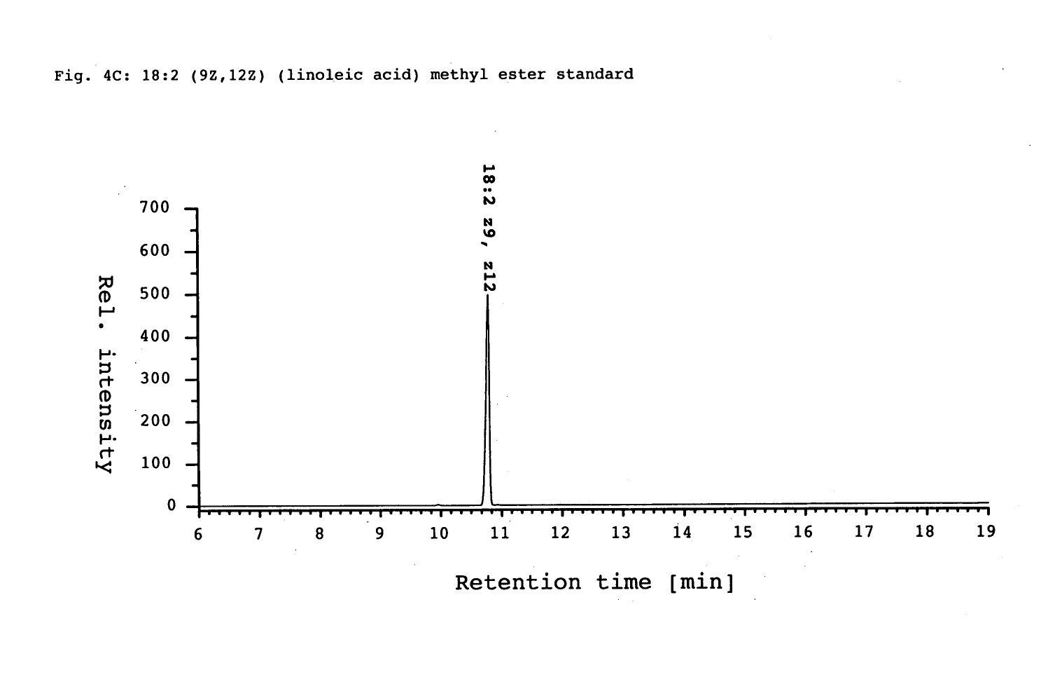

FIG. 4 shows the formation of linoleic acid in yeast cells transformed with the Punica granatum Δ-12-desaturase. FIG. 4A shows the gas chromatogram of the lipid extracts from yeast cells transformed with the blank vector pESC-Leu. The cells were grown for 240 hours at 16° C. as described in Example 4. The gas chromatogram shows no FAMEs with a retention time of linoleic acid. The content of oleic acid (retention time=10.0 min) amounts to xxx % of the total fatty acids. FIG. 4B shows the gas chromatogram of the lipid extracts from yeast cells transformed with pESC-PuFAD12. Again, the cells were grown for 240 hours at 16° C. as described in Example 4. The gas chromatogram shows a pronounced peak with a retention time of 10.75 min, which is not found in the control batch (cf. FIG. 4A) and has the same retention time as linoleic acid (cf. FIG. 4C). The content of oleic acid amounts to 85%, that is to say 15% lower than in control yeast cells. These results show that the expression of PuFAD12 in yeast leads to the conversion of oleic acid into linoleic acid. Coexpression of PuFAD12 and PuFADX in yeast therefore leads to the formation of punicic acid, even without added linoleic acid.

### Example 6

### Expression of Punicic Acid Desaturase in Plants

The expression of Punica granatum punicic acid desaturase in transgenic plants is advantageous in order to increase the punicic acid content in these plants. To this end, the PuFADX or PuFADX2 cDNA was cloned into binary vectors and transferred into Arabidopsis thaliana, Nicotiana tabacum, Brassica napus and Linum usitatissimum via Agrobacterium-mediated DNA transfer. Expression of the calendulic acid desaturase cDNA was under the control of the constitutive CaMV 35 S promoter or of the seed-specific USP promoter.

Arabidopsis is particularly suitable as model plant since it has a short generation cycle and sufficient amounts of linoleic acid, the substrate of PuFADX or PuFADX2 for the production of punicic acid, and also sufficient amounts of oleic acid, the substrate of PuFADX for the production of conjudienoic fatty acids such as CLA.

Tobacco and high-linoleic acid varieties of linseed, such as the variety Linola, are oilseed crops with a high linoleic acid content and therefore particularly suitable for the heterologous expression of PuFADX or PuFADX2 since linoleic acid constitutes the substrate of PuFADX or PuFADX2 for the formation of punicic acid.

Oilseed rape is an oilseed crop with a high oleic acid content and therefore particularly suitable for converting oleic acid into conjudienoic fatty acids such as CLA by expressing PuFADX or PuFADX2 and accumulating the former. Moreover, the expression of PuFAD12 in oilseed rape allows an increase in the linoleic acid content and the coexpression of PuFAD12 and PuFADX or PuFADX2 allows the accumulation of punicic acid.

The expression vectors used were the vector pBinAR (Höfgen and Willmitzer, Plant Science, 66, 1990; 221-230) and the pBinAR derivative pBinAR-USP, in which the V. faba USP promoter had been substituted for the CaMV 35 S promoter. The vectors pGPTV and pGPTV-USP were also used. For recloning, the calendulic acid desaturase cDNA had to be excised from the vector pGEM-T and cloned into pBinAR or pBinAR-USP.

The resulting plasmids pBinAR-PuFADX, pBinAR-USP-PuFADX, pGPTV-PuFADX, pGPTV-USP-PuFADX, pBinAR-PuFAD12, pBinAR-USP-PuFAD12, pGPTV-PuFAD12, pGPTV-USP-PuFAD12 were transformed into Agrobacterium tumefaciens (Höfgen and Willmitzer, Nucl. Acids Res., 16, 1988: 9877). A. thaliana was transformed by “floral dip” (Clough and Bent, Plant Journal, 16, 1998: 735-743), while N. tabacum was transformed by coculturing tobacco leaf sections with transformed A. tumefaciens cells, and linseed and oilseed rape were transformed by coculturing hypocotyl sections with transformed A. tumefaciens cells.

Expression of the PuFADX and PuFAD12 genes in transgenic Arabidopsis, tobacco, oilseed rape and linseed plants were analyzed by Northern blot. Selected plants were analyzed for their content of punicic acid or other conjugated fatty acids such as CLA in seed oil.

To achieve a seed-specific expression of PuFADX, PuFADX2 and PuFAD12, the napin promoter may also be used analogously to the USP promoter.

The PuFADX and PuFADX2 full-length cDNAs were amplified under the control of the USP promoter and the OCS terminator with the primers M and N for expression in plants. This was performed using the Expand High Fidelity PCR System (Roche Diagnostics).
Primer M: 5′-GGA TCC ATG GGA GCT GAT GGA ACA ATG TCT C-3′ forward primer (with BamHI cleavage site)
Primer N: 5′-GGG CCC ATT CAG AAC TTG CTC TTG AAC CAT AG-3′ reverse primer (with ApaI cleavage site)

The PCR reactions were composed as follows:

| | | |
|---|---|---|
| dNTP mix: | 1 | μl (10 mM) |
| 5′ primer: | 4 | μl (10 μM) |
| 3′ primer: | 4 | μl (10 μM) |
| template: | 3 | μl (cDNA marathon bank, diluted 1:50) |
| polymerase: | 0.5 | μl (3.5 U/μl) |
| 10 × buffer 2: | 5 | μl |
| water | 32.5 | μl |
| total volume: | 50 | μl |

The PCR was carried out with the following program:

| | | |
|---|---|---|
| 1. | 2 min | 94° C. |
| 2. | 30 sec | 94° C. |
| 3. | 30 sec | 50° C. |
| 4. | 2 min | 72° C. |
| 5. | 10 × 2.-4. | |
| 6. | 30 sec | 94° C. |
| 7. | 30 sec | 50° C. |
| 8. | 2 min | 72° C., time increment 5 sec per cycle |
| 9. | 15 × 5.-7. | |
| 10. | 5 min | 72° C. |

The PCR products were cloned into the vector pUC19-USP-OCS2 via the cleavage sites which had been introduced and transformed into E.coli-XL1-Blue cells. The insert DNA was sequenced as a double strand using a 373 DNA sequencer (Applied Biosystems). After amplification of the plasmid DNA, the expression cassette (USP-PuFADX-OCS and USP-PuFADX2-OCS) was excised using the restriction enzymes SacI. After T4-polymerase treatment for generating smooth ends and ligation into pPTV-bar (HindIII-cut, likewise with smooth ends by means of T4-polymerase), the construct pPTV-bar-USP-PuFADX-OCS-DNA was generated. The construct for PuFADX2 was prepared analogously. This plasmid was transformed into competent agrobacterial cells (Agrobacterium tumefaciens EHA105) and tobacco plants (Nicotiana tabacum SR1) following a standard method (Horsch et al. (1985) Science, 269, 1985: 1229-1231), and transgenic tobacco plants were regenerated.

The seeds of transgenic plants were harvested and 10 mg of seeds were homogenized in 405 μl of methanol:toluene (2:1) and extracted with 150 μl of 0.5 M sodium methoxide. The seed material was comminuted as finely as possible in a pestle and mortar and subsequently incubated for 20 minutes at room temperature, with shaking. Thereafter, 0.5 ml of 1 M NaCl solution and 0.5 ml of n-heptane were added and the mixture was incubated for 5 minutes at room temperature for extraction. After phase separation by centrifugation (10 min, 4 000 rpm, 4° C.), the heptane supernatant was transferred into a reaction vessel and evaporated under nitrogen. The residue was taken up in 3 times 300 μl of hexane and again evaporated under nitrogen. The residue was taken up in 40 μl of MeCN and the sample was analyzed by means of GC or GC/MS.

To carry out the GC analysis of the fatty acid methyl esters (FAMEs), 7 μl of the sample (in MeCN) were transferred into a test tube and 1 μl was injected. The GC analysis was carried out using an HP-DB23 column (Crosslinked PEG; 30 m×0.32 mm×0.5 μm film thickness) at a flow rate of 1.5 ml/min. Helium acted as the carrier gas. The injection temperature was 220° C. The following temperature gradient was applied: 1 min 150° C., 150° C. to 200° C. (15° C./min), 200° C. to 250° C. (2° C./min), 5 min 250° C. The FAMEs were detected via a flame ionization detector (FID) at 275° C.

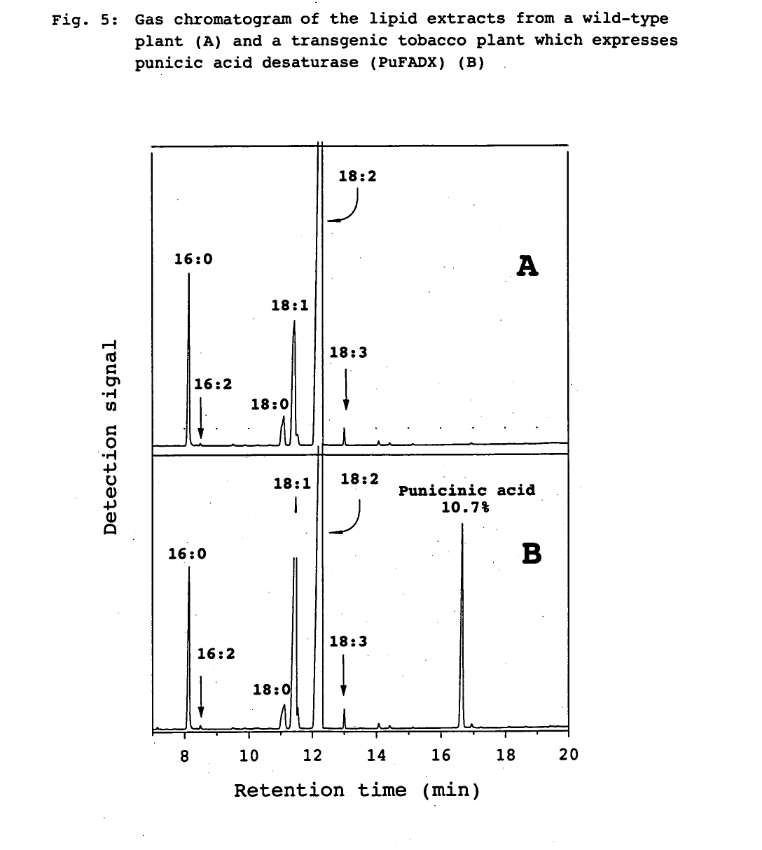

FIG. 5 shows the production of punicic acid in tobacco seeds which were transformed with the Punica granatum punicic acid desaturase (PuFADX). The experiments with PuFADX2 gave the same results.

The results shown in FIG. 5 demonstrate that punicic acid desaturase in tobacco plants leads to the formation of punicic acid. Since in tobacco seeds the fatty acids are mostly bound in triacyl glycerides, it must be assumed that most of the punicic acid detected was bound in the triacyl glycerides of the tobacco seeds. FIG. 5.A shows the control without PuFADX desaturase. FIG. 5.B shows the synthesis of punicic acid with the aid of PuFADX desaturase.

Corresponding results can be obtained in tobacco with the PuFADX2 gene.

TABLE I
Fatty acid composition of the transgenic tobacco seeds analyzed (F71 clones No. 1 to 50)
which express punicic acid desaturase, in comparison with wild-type tobacco plants
(SNNWT/1 and SNNWT/2)

| 16:0 | 16:1 | 18:0 | 18:1-(9Z) | 18:1-(11Z) | 18:2 | 18:3 | 18:3Pu | |
|---|---|---|---|---|---|---|---|---|
| FA % | FA % | FA % | FA % | FA % | FA % | FA % | FA % | Pl. No. |
| 8.9 | 0.1 | 3.0 | 12.4 | 0.7 | 74.3 | 0.6 | — | SNNWT/1 |
| 8.8 | 0.1 | 3.0 | 12.1 | 0.8 | 74.5 | 0.6 | — | SNNWT/2 |
| 8.7 | 0.1 | 2.7 | 11.9 | 0.9 | 73.0 | 1.0 | 1.7 | F71/1 |
| 9.1 | 0.1 | 3.2 | 13.4 | 0.8 | 71.1 | 0.7 | 1.6 | F71/3 |
| 8.0 | 0.1 | 2.9 | 18.2 | 0.8 | 62.5 | 0.5 | 6.9 | F71/4 |
| 8.1 | 0.1 | 2.5 | 15.7 | 0.9 | 65.4 | 0.7 | 6.6 | F71/35 |
| 8.7 | 0.1 | 2.7 | 11.4 | 0.7 | 74.2 | 0.7 | 1.4 | F71/52 |
| 8.8 | 0.1 | 2.8 | 13.0 | 0.9 | 72.1 | 0.8 | 1.5 | F71/2 |
| 8.7 | 0.1 | 2.5 | 11.0 | 0.9 | 74.5 | 1.0 | 1.3 | F71/28 |
| 8.5 | 0.1 | 2.4 | 11.4 | 0.9 | 74.3 | 1.0 | 1.3 | F71/31 |
| 7.1 | 0.1 | 2.4 | 20.6 | 0.8 | 57.7 | 0.6 | 10.7 | F71/33 |
| 8.1 | 0.1 | 2.3 | 13.1 | 0.7 | 70.7 | 0.9 | 4.0 | F71/46 |
| 8.5 | 0.1 | 2.8 | 17.1 | 0.6 | 64.4 | 0.5 | 6.0 | F71/4 |
| 8.9 | 0.1 | 2.8 | 13.5 | 0.6 | 71.7 | 0.8 | 1.7 | F71/11 |
| 8.8 | 0.1 | 2.5 | 11.7 | 0.6 | 73.8 | 1.0 | 1.5 | F71/24 |
| 9.0 | 0.1 | 2.6 | 13.0 | 0.5 | 72.3 | 0.8 | 1.6 | F71/29 |
| 9.4 | 0.1 | 2.7 | 11.0 | 0.0 | 75.7 | 1.0 | 0.1 | F71/30 |
| 8.9 | 0.1 | 2.8 | 13.1 | 0.5 | 71.7 | 0.9 | 2.0 | F71/34 |
| 9.3 | 0.1 | 2.7 | 12.0 | 0.0 | 73.9 | 0.8 | 1.1 | F71/36 |
| 9.1 | 0.1 | 2.7 | 12.3 | 0.4 | 73.4 | 0.9 | 1.3 | F71/40 |
| 9.1 | 0.1 | 2.8 | 13.2 | 0.5 | 71.8 | 0.8 | 1.6 | F71/45 |
| 7.7 | 0.1 | 2.4 | 20.9 | 0.5 | 57.4 | 0.6 | 10.4 | F71/48 |
| 8.7 | 0.1 | 2.5 | 13.3 | 0.5 | 71.5 | 0.9 | 2.6 | F71/49 |
| 9.1 | 0.1 | 2.6 | 11.2 | 0.4 | 74.2 | 0.9 | 1.4 | F71/50 |

Table I shows clearly that all desaturase clones (F71 clones) synthesize punicic acid.

1. An isolated nucleic acid that encodes a polypeptide with a desaturase activity, wherein said nucleic acid contains a sequence selected from the group consisting of:
the sequence of SEQ ID NO: 2;
the sequence of SEQ ID NO: 7;
a sequence derived from the amino acid sequence of SEQ ID NO: 3 or SEQ ID NO: 8 by back translation of the respective amino acid sequences, owing to the degeneracy of the genetic code; and
a derivative of the sequence of SEQ ID NO: 2 or SEQ ID NO: 7 wherein said derivative encodes a peptide that has at least 75 % identity at the amino acid level to said polypeptide, wherein the desaturase activity of the peptide is not substantially reduced as compared with the desaturase activity of said polypeptide.

2. An amino acid sequence encoded by the nucleic acid of claim 1.

3. The amino acid sequence of claim 2, which contains the sequence of SEQ ID NO: 3 or SEQ ID NO: 8.

4. A nucleic acid construct comprising the nucleic acid of claim 1 linked to one or more regulatory signal sequences.

5. A vector comprising the nucleic acid construct of claim 4.

6. An organism comprising the vector of claim 5.

7. The organism of claim 6, which is a plant, a microorganism or an animal.

8. A transgenic plant comprising the nucleic acid of claim 1.

9. A process for production of oils or triglycerides with an increased content of unsaturated fatty acids, which comprises:
introducing the nucleic acid of claim 1 to an oil-producing organism;
culturing said organism; and
isolating the oils or triglycerides.

10. The process of claim 9, further comprising liberating said oils or triglycerides from said organism.

11. The process of claim 9, further comprising introducing at least one further nucleic acid to the organism, wherein said at least one further nucleic acid encodes a polypeptide with a desaturase activity, which is functional or non-functional, and said at least one further nucleic acid contains a sequence selected from the group consisting of:
a sequence that encodes a Δ-5-desaturase, a Δ-6-desaturase, a Δ-8-desaturase or Δ-12-desaturase;
the sequence of SEQ ID NO: 5;
a sequence derived from the amino acid sequence of SEQ ID NO: 6 by back translation of said amino acid sequence, owing to the degeneracy of the genetic code; and
a derivative of the sequence of SEQ ID NO: 5 wherein said derivative encodes a peptide that has at least 90% identity with the amino acid sequences of SEQ ID NO: 6 and the desaturase activity of said peptide is not substantially reduced as compared to the desaturase activity of said polypeptide.

12. The process of claim 9, wherein the unsaturated fatty acids have an increased content of punicic acid.

13. The process of claim 9, wherein the unsaturated fatty acids have an increased content of octadecaconjudienoic fatty acids.

14. The process of claim 9, wherein the unsaturated fatty acids have an increased content of octadecaconjutetraenoic fatty acids.

15. The nucleic acid of claim 1, wherein the polypeptide has a functional desaturase activity.

16. The process of claim 11, wherein the polypeptide has a functional desaturase activity.

17. The process of claim 9, wherein the organism is a plant or a microorganism.

18. A composition comprising oils or triglycerides with an increased content of unsaturated fatty acids prepared by the process of claim 9.

19. A composition comprising oils or triglycerides with an increased content of unsaturated fatty acids prepared by the process of claim 11.

20. A process for generating transgenic plants comprising introducing the construct of claim 4 to a plant.

21. A process for isolating a genomic sequence via homology screening comprising screening a genome with the nucleic acid of claim 1 or a fragment thereof.

22. A process for production of foods, animal feed, cosmetics or pharmaceuticals comprising introducing the nucleic acid of claim 1 to an organism.

23. An isolated nucleic acid sequence that encodes a protein that converts a fatty acid of structure I 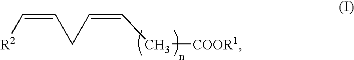
and has two double bonds separated from each other by a methylene group, to a triunsaturated fatty acid of structure II 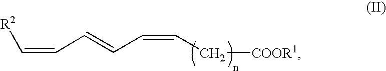
in which the three double bonds of the fatty acid are conjugated and the variables in structure I and structure II have the following meanings:
R¹=hydrogen, substituted or unsubstituted, unsaturated or saturated, branched or unbranched C₁-C₁₀-alkyl-, 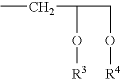
R²=substituted or unsubstituted, unsaturated or saturated C₁-C₉-alkyl-,
R³ and R⁴ independently of one another, hydrogen, substituted or unsubstituted, saturated or unsaturated, branched or unbranched C₁-C₂₂-alkylcarbonyl- or phospho-, and
n=1 to 14.

24. An isolated nucleic acid that encodes a polypeptide with a desaturase activity, wherein said nucleic acid contains a sequence selected from the group consisting of:
the sequence of SEQ ID NO: 5;
a sequence derived from the amino acid sequence of SEQ ID NO: 6 by back translation of said amino acid sequence, owing to the degeneracy of the genetic code;
a derivative of the sequence of SEQ ID NO: 5 that encodes a peptide which has at least 90% identity at the amino acid level with the amino acid sequence of SEQ ID NO: 6, wherein the desaturase activity of said peptide is not substantially reduced as compared with the desaturase activity of said polypeptide.

25. An amino acid sequence encoded by the nucleic acid of claim 24.

26. A nucleic acid construct comprising the nucleic acid of claim 24, linked to one or more regulatory signal sequences.

27. A vector comprising the nucleic acid construct of claim 26.

28. An organism comprising the vector of claim 27.

29. A transgenic plant comprising the nucleic acid of claim 24.

30. A process for isolating a genomic sequence via homology screening comprising screening a genome with the nucleic acid of claim 24 or a fragment thereof.

31. The process of claim 11, wherein the nucleic acid encodes a non-functional polypeptide.

32. The process of claim 31, wherein the nucleic acid is an antisense nucleic acid.

33. An isolated nucleic acid that encodes a polypeptide with a non-functional desaturase activity, wherein said nucleic acid contains a sequence selected from the group consisting of:
the sequence of SEQ ID NO: 2;
the sequence of SEQ ID NO: 7;
a sequence derived from the amino acid sequence of SEQ ID NO: 3 or SEQ ID NO: 8 by back translation of the respective amino acid sequences, owing to the degeneracy of the genetic code; and
a derivative of the sequence of SEQ ID NO: 2 or SEQ ID NO: 7 wherein said derivative encodes a peptide that has at least 75 % identity at the amino acid level to said polypeptide.

34. The nucleic acid of claim 33, which is an antisense nucleic acid.

35. A nucleic acid construct comprising the nucleic acid of claim 33 linked to one or more regulatory signal sequences.

36. A vector comprising the nucleic acid construct of claim 35.

37. An organism comprising the vector of claim 36.

38. The organism of claim 37, which is a plant, a microorganism or an animal.

39. A transgenic plant comprising the nucleic acid of claim 33.

40. A process for production of oils or triglycerides with an increased content of unsaturated fatty acids, which comprises:
introducing the nucleic acid of claim 33 to an oil-producing organism;
culturing said organism; and
isolating the oils or triglycerides.

41. The process of claim 40, further comprising liberating said oils or triglycerides from said organism.

42. The process of claim 40, further comprising introducing at least one further nucleic acid to the organism, wherein said at least one further nucleic acid encodes a polypeptide with functional or non-functional desaturase activity and said at least one further nucleic acid contains a sequence selected from the group consisting of:
a sequence that encodes a Δ-5desaturase, a Δ-6-desaturase, a Δ-8-desaturase or Δ-12-desaturase;
the sequence of SEQ ID NO: 5;
a sequence derived from the amino acid sequence of SEQ ID NO: 6 by back translation of said amino acid sequence, owing to the degeneracy of the genetic code; and
a derivative of the sequence of SEQ ID NO: 5 wherein said derivative encodes a peptide that has at least 90% identity with the amino acid sequences of SEQ ID NO: 6.

43. The process of claim 40, wherein the oils or triglycerides have an increased content of punicic acid.

44. The process of claim 40, wherein the oils or triglycerides have an increased content of octadecaconjudienoic fatty acids.

45. The process of claim 40, wherein the oils or triglycerides have an increased content of octadecaconjutetraenoic fatty acids.

46. A process for production of oils or triglycerides with an increased content of saturated fatty acids, which comprises:
introducing a nucleic acid to an oil-producing organism wherein said nucleic acid encodes a polypeptide with a non-functional desaturase activity, wherein said nucleic acid contains a sequence selected from the group consisting of:
the sequence of SEQ ID NO: 2;
the sequence of SEQ ID NO: 7;
a sequence derived from the amino acid sequence of SEQ ID NO: 3 or SEQ ID NO: 8 by back translation of the respective amino acid sequences, owing to the degeneracy of the genetic code; and
a derivative of the sequence of SEQ ID NO: 2 or SEQ ID NO: 7
wherein said derivative encodes a peptide that has at least 75 % identity at the amino acid level to said polypeptide;
culturing said organism; and
isolating said oils or triglycerides.

47. The process of claim 46, wherein the nucleic acid is an antisense nucleic acid.

48. The process of claim 46, further comprising liberating said oils or triglycerides.

49. The process of claim 46, wherein the oil-producing organism is a plant or a microorganism.

50. A composition comprising oils or triglycerides with an increased content of unsaturated fatty acids prepared by the process of claim 46.

51. A process for isolating a genomic sequence via homology screening comprising screening a genome with the nucleic acid of claim 33 or a fragment thereof.

52. A process for production of foods, animal feed, cosmetics or pharmaceuticals comprising introducing the nucleic acid of claim 33 to an organism.

53. An isolated nucleic acid that encodes a polypeptide with a non-functional desaturase activity, wherein said nucleic acid contains a sequence selected from the group consisting of:
the sequence of SEQ ID NO: 5;
a sequence derived from the amino acid sequence of SEQ ID NO: 6 by back translation of said amino acid sequence, owing to the degeneracy of the genetic code; and
a derivative of the sequence of SEQ ID NO: 5 that encodes a peptide which has at least 90% identity at the amino acid level with the amino acid sequence of SEQ ID NO: 6.

54. The nucleic acid of claim 53, which is an antisense nucleic acid.

55. A nucleic acid construct comprising the nucleic acid sequence of claim 53, which is linked to one or more regulatory signal sequences.

56. A vector comprising the nucleic acid construct of claim 55.

57. An organism comprising the vector of claim 56.

58. A transgenic plant comprising the nucleic acid of claim 53.


SEQUENCE LISTING (8 sequences)

SEQ ID NO: 1 - DNA, 586 bp, Punica granatum
tgggtgattgctcatgagtgtggccaccaggctttcagcaattacggctgggtaaatgatgcagtgggcttcttcctccacacatcgctcctcgtcccatactttccatttaagtacagccaccgtcgccaccactccaacaccaactccgtcgagcatgacgaggtatttgtcccgaggcacaaggatggtgttcagtggtattacaggttcttcaacaacacccctggccgagttctaaccctaacgctgactctactggtgggctggccatcatacctggcattcaatgcgtcgggtaggccctatgatggcttcgcatcccattacaaccccaatgctcagatattcaacttgagagagcggttctgggtccacgtctcgaatatcgggattttagccatctactacatcctctaccggctagccaccacgaaaggcctcccatggcttctcagcatctacggagtcccagtcctcatattaaatgcattcgtggtgttaataaccttccttcaacactctcatcctgcactcccccactacaactcagacgaatgggactggctgagaggggcacttagcc

SEQ ID NO: 2 - DNA, 1464 bp, Punica granatum
  CDS (131)..(1252)
ccatcctaatacgactcactatagggctcgagcggccgcccgggcaggtggggggcggaaagtgcttttccaacggcggagatcaaggaaaagtggcaaagtggaaagcacctactactacacccaaaacatgggagctgatggaacaatgtctcctgtcctaaccaaaagaaggcccgaccaagagatcaacaaactcgacataaagcctaaccatgaggtcgacattgcccgaagagcccctcactcgaagccgcccttcaccttgagcgacctccggagcgcaatcccgccgcactgcttccaccgctcgctcctcatgtcctcatcgtacctcatccgcgacttcgccctagccttcctcttttaccactctgccgtcacttacatcccgctccttccgaaaccacttgcctgcatggcttggccggtctactggttcttgcagggatcgaacatgctcggcatctgggtcattgcccacgagtgcggccaccaggctttcagcaattacggctgggtaaatgatgcagtgggcttcttcctccacacatcgctcctcgtcccatactttccatttaagtacagccaccgtcgccaccactccaacaccaactccgtcgagcatgacgaggtatttgtcccgaggcacaaggatggtgttcagtggtattacaggttcttcaacaacacccctggccgagttctaaccctaacgctgactctactggtgggctggccatcatacctggcattcaatgcgtcgggtaggccctatgatggcttcgcatcccattacaaccccaatgctcagatattcaacttgagagagcggttctgggtccacgtctcgaatatcgggattttagccatctactacatcctctaccggctagccaccacgaaaggcctcccatggcttctcagcatctacggagtcccagtcctcatattaaatgcattcgtggtgttaataaccttccttcaacactctcatcctgcactcccccactacaactcagacgaatgggactggctgagaggggcactagccacagttgatcgagattacggttttctaaacgaggttttccacgacataacagacactcacgtgatccatcacctcttcccaacaatgccccattacaatgccaaggaggctactgtgtccataaggtcaatcttgaaggactactacaagtttgataggacgcccatttggagagcattgtggagggaggccaaggagtgattgtacgtagaagctgatggcactggcagcaaaggggtgctatggttcaagagcaagttctgaatgagtgttattagaatcgaacctaatatcgagccagtcgagatatgagttcataggttcaagggtccaactaaggttcaatggatcaaaccgtatgtttttgttaaaaatcaaataaataatttatattatttaaaaaaaaaaaa

SEQ ID NO: 3 - protein, 374 aa, Punica granatum
MGADGTMSPVLTKRRPDQEINKLDIKPNHEVDIARRAPHSKPPFTLSDLRSAIPPHCFHRSLLMSSSYLIRDFALAFLFYHSAVTYIPLLPKPLACMAWPVYWFLQGSNMLGIWVIAHECGHQAFSNYGWVNDAVGFFLHTSLLVPYFPFKYSHRRHHSNTNSVEHDEVFVPRHKDGVQWYYRFFNNTPGRVLTLTLTLLVGWPSYLAFNASGRPYDGFASHYNPNAQIFNLRERFWVHVSNIGILAIYYILYRLATTKGLPWLLSIYGVPVLILNAFVVLITFLQHSHPALPHYNSDEWDWLRGALATVDRDYGFLNEVFHDITDTHVIHHLFPTMPHYNAKEATVSIRSILKDYYKFDRTPIWRALWREAKE

SEQ ID NO: 4 - DNA, 567 bp, Punica granatum
  misc_feature (66)..(66): n is a, c, g, or t
tgggtgatcgctcacgagtgggggcaccatgcgtttagcgactaccagtgggtggacgactgtgtnggnctggtactgcactcagcgctcctcgttccgtacttctcctggaagtacagccaccgccggcaccactccaacacgggctcgntggagcgtgacgaggttttcgtccccaagcccaagtccaagatgccgtggttctccaagtacctgaacaacccgccaggccgagtcatgacgctgattgtgaccctgaccctgggctggccgttgtacttggcattgaacgtctctggctggccctatgacaggttcgcttgccactttgacccgtatggcccgatctacaccgacagggagcgtctccagatctacatttctgatgtagggatcatggccgccacgtacacgctgtacaagatcgcagcagcccgtggcctggcctggttggtttgtgtatatggtgtccctctcctgatcgtgaacgcattcctggtcacgatcacctacctccancacacccacccggcccttccccactacgactcctccggatggaattgn

SEQ ID NO: 5 - DNA, 1398 bp, Punica granatum
  CDS (94)..(1254)
  misc_feature (556)..(556): n is a, c, g, or t
tctcctaatacgactcactatagggctcgagcggccgcccgggcaggttatccctccgaggtccggagaaggtgagctctcagggacgacgcgatgggagccggtggaagaatgacggtcccgaacaagtgggaaggcgagggagacgagaagagccagaagccggtccaaagggttccctccgcaaagccaccattcacactaagcgagatcaagaaggccatcccgccacattgcttcaaacgctccctcctcaagtccttctcttatgtcctctatgacctcactttggtggccatcttctattacgttgctaccacttacatcgacgccctcccgggtccactacgctacgcggcctggcccgtgtactgggccctgcaggggtgcgtgctcacgggtgtctgggtcatagcccacgagtgcgggcaccatgcgtttagcgactaccagtgggtggacgactgtgtcggcctggtactgcactcagcgctcctcgttccgtacttctcctggaagtacagccaccgccggcaccactccaacacgggctcgntggagcgtgacgaggttttcgtncccaagcccaagtccaagatgccgtggttctccaagtacctgaacaacccgccaggccgagtcatgacgctgattgtgaccctgaccctgggctggccgttgtacttggcattgaacgtctctggctggccctatgacaggttcgcttgccactttgacccgtatggcccgatctacaccgacagggagcgtctncagatctacatttctgatgtagggatcatggccgccacgtacacgctgtacaagatcgcagcagcccgtggcctggcctggttggtttgtgtatatggtgtccctctcctgatcgtgaacgcattcctggtcacgatcacctacctccagcacacccacccggcccttccccactatgactcgtcggaatgggactggctcaggggggcactggcaacagcggaccgagactacgggatcctcaacaaggtcttccacaacataaccgacacgcatgtcgcccaccacctcttctccaccatgccgcactaccacgcgatggaggctaccaaagcgatcaagccgatactgggagactactaccagttcgacgggactccggtatacaaagcgatgtggagagaggctagggagtgcctgtacgtggagcccgacgacggggccaacagtaagggggttttctggtacaagaagaacctctgatcaccatttaccacattgctggtcgcaagttaatttcaaggtcataccgaacaaaacgaattagaatctttagctttagggtgtcctctctctctctcttgagcgagcgaataaactagtacttgatcggaaaaaaaaaaa

SEQ ID NO: 6 - protein, 387 aa, Punica granatum
  misc_feature (155)..(155): Xaa can be any naturally occurring amino acid
MGAGGRMTVPNKWEGEGDEKSQKPVQRVPSAKPPFTLSEIKKAIPPHCFKRSLLKSFSYVLYDLTLVAIFYYVATTYIDALPGPLRYAAWPVYWALQGCVLTGVWVIAHECGHHAFSDYQWVDDCVGLVLHSALLVPYFSWKYSHRRHHSNTGSXERDEVFXPKPKSKMPWFSKYLNNPPGRVMTLIVTLTLGWPLYLALNVSGWPYDRFACHFDPYGPIYTDRERXQIYISDVGIMAATYTLYKIAAARGLAWLVCVYGVPLLIVNAFLVTITYLQHTHPALPHYDSSEWDWLRGALATADRDYGILNKVFHNITDTHVAHHLFSTMPHYHAMEATKAIKPILGDYYQFDGTPVYKAMWREARECLYVEPDDGANSKGVFWYKKNL

SEQ ID NO: 7 - DNA, 1188 bp, Punica granatum
  CDS (1)..(1188)
atgggagctgatggaacaatgtctcctgtcctaaccaaaagaaggcccgaccaagagatcaacaaactcgacataaagcctaaccatgaggtcgacattgcccgaagagcccctcactcgaagccgcccttcaccttgagcgacctccggagcgcaatcccgccgcactgcttccaccgctcgctcctcatgtcctcatcgtacctcatccgcgacttcgccctagccttcctcttttaccactctgccgtcacttacatcccgctccttccgaaaccacttgcctgcatggcttggccggtctactggttcttgcagggatcgaacatgctcggcatctgggtcattgcccacgagtgcggccaccaggctttcagcaattacggctgggtaaatgatgcagtgggcttcttcctccacacatcgctcctcgtcccatactttccatttaagtacagccaccgtcgccaccactccaacaccaactccgtcgagcatgacgaggtatttgtcccgaggcacaaggatggtgttcagtggtattacaggttcttcaacaacacccctggccgagttctaaccctaacgctgactctactggtgggctggccatcatacctggcattcaatgcgtcgggtaggccctatgatggcttcgcatcccattacaaccccaatgctcagatattcaacttgagagagcggttctgggtccacgtctcgaatatcgggattttagccatctactacatcctctaccggctagccaccacgaaaggcctcccatggcttctcagcatctacggagtcccagtcctcatattaaatgcattcgtggtgttaataaccttccttcaacactctcatcctgcactcccccactacaactcagacgaatgggactggctgagaggggcactagccacagttgatcgagattacggttttctaaacgaggttttccacgacataacagacactcacgtgatccatcacctcttcccaacaatgccccattacaatgccaaggaggctactgtgtccataaggtcaatcttgaaggactactacaagtttgataggacgcccatttggagagcattgtggagggaggccaaggagtgcttgtacgtagaagctgatggcactggcagcaaaggggtgctatggttcaagagcaagttctga

SEQ ID NO: 8 - protein, 395 aa, Punica granatum
MGADGTMSPVLTKRRPDQEINKLDIKPNHEVDIARRAPHSKPPFTLSDLRSAIPPHCFHRSLLMSSSYLIRDFALAFLFYHSAVTYIPLLPKPLACMAWPVYWFLQGSNMLGIWVIAHECGHQAFSNYGWVNDAVGFFLHTSLLVPYFPFKYSHRRHHSNTNSVEHDEVFVPRHKDGVQWYYRFFNNTPGRVLTLTLTLLVGWPSYLAFNASGRPYDGFASHYNPNAQIFNLRERFWVHVSNIGILAIYYILYRLATTKGLPWLLSIYGVPVLILNAFVVLITFLQHSHPALPHYNSDEWDWLRGALATVDRDYGFLNEVFHDITDTHVIHHLFPTMPHYNAKEATVSIRSILKDYYKFDRTPIWRALWREAKECLYVEADGTGSKGVLWFKSKF